(19) United States
(12) Patent Application Publication
(10) Pub. No.: US 2019/0008828 A1
(43) Pub. Date: Jan. 10, 2019

(54) METHODS FOR INHIBITING HUMAN IMMUNODEFICIENCY VIRUS (HIV) RELEASE FROM INFECTED CELLS

(71) Applicant: THE UNITED STATES OF AMERICA, AS REPRESENTED BY THE SECRETARY, DEPARTMENT OF HEALTH AND HUMAN SERV, Bethesda, MD (US)

(72) Inventors: Peter D. SUN, Rockville, MD (US); Joseph P. KONONCHIK, Rockville, MD (US); Joanna L. IRELAND, Rockville, MD (US)

(73) Assignee: The U.S.A., as represented by the Secretary Department of Health and Human Services, Bethesda, MD (US)

(21) Appl. No.: 16/065,854

(22) PCT Filed: Dec. 27, 2016
(86) PCT No.: PCT/US2016/068713

§ 371 Date: Jun. 25, 2018

Related U.S. Application Data
(60) Provisional application No. 62/271,726, filed on Dec. 28, 2015.

(51) Int. Cl. A61K 31/381; A61K 45/06; C12N 5/0783; A61P 31/18; C07K 16/28

(52) U.S. Cl. CPC A61K 31/381; A61K 45/06; C12N 5/0636; C12N 2501/998; C07K 16/2854; C12N 2501/999; A61P 31/18

(57) ABSTRACT

The finding that human immunodeficiency virus (HIV) envelope glycans bind CD62L (L-selectin) on central memory T cells is described. HIV infection is also shown to induce shedding of CD62L and this shedding is required for efficient release of HIV from infected cells. Methods of inhibiting HIV release from infected cells using inhibitors of CD62L sheddase are described. Methods of treating HIV infection with a CD62L sheddase, such as in combination with antiretroviral therapy, is also described.

### CROSS REFERENCE TO RELATED APPLICATIONS

This application claims the benefit of U.S. Provisional Application No. 62/271,726, file Dec. 28, 2015, which is herein incorporated by reference in its entirety.

### FIELD

This disclosure concerns methods of inhibiting human immunodeficiency virus (HIV) release from infected cells using inhibitors that target CD62L sheddase(s). This disclosure further concerns methods of treating HIV by inhibiting HIV release and spread.

### BACKGROUND

While human immunodeficiency virus type 1 (HIV-1) infects all CD4⁺ T cells, the virus exhibits a clear preference for some subsets of CD4⁺ T cells (Brenchley et al., J Virol 78, 1160-1168, 2004; Ostrowski et al., J Virol 73, 6430-6435, 1999; Douek et al., Nature 417, 95-98, 2002; Schnittman et al., Proc Natl Acad Sci USA 87, 6058-6062, 1990), particularly central memory CD4⁺ T cells (T_{CM}) (Holl et al., Arch Virol 152, 507-518, 2007). The persistence of HIV-1 infection of T_{CM} suggests that this subset constitutes a major viral reservoir, even under antiretroviral therapy (ART) (Chomont et al., Nat Med 15, 893-900, 2009; Lambotte et al., Aids 16, 2151-2157, 2002; Hua et al., Immunol Invest 41, 1-14, 2012). The loss of these memory T cells is profound in HIV-1-infected individuals; it is associated with dysfunctional immune responses and disease progression, and its recovery under ART treatment was shown to correlate with a better clinical outcome (Yang et al., PLoS One 7, e49526, 2012; Letvin et al., Science 312, 1530-1533, 2006; Potter et al., J Virol 81, 13904-13915, 2007). However, the mechanism for the underlying importance and preferential depletion of central memory CD4⁺ T cells in HIV biology is not well understood. The reason for the preferential replication of HIV-1 in central memory CD4⁺ T cells is not evident based on levels of expression of known co-receptors, CD4, and chemokine receptors. Despite the success of ART in controlling HIV-1 in infected individuals, treatment is less effective at eliminating HIV-1 viral reservoirs. The nature of HIV-1 reservoirs and the factors controlling their size and release are a major research focus for achieving a cure for HIV/AIDS.

### SUMMARY

Described herein is the finding that shedding of CD62L (L-selectin) on T cells is required for the efficient release of HIV from infected cells.

Provided are methods of inhibiting HIV release from an infected cell. In some embodiments, the method includes contacting the cell with an inhibitor of CD62L shedding. In some embodiments, the method is an in vitro or ex vivo method. In other embodiments, the method is an in vivo method that includes administering an inhibitor of CD62L to a subject infected with HIV.

Also provided are methods of treating a subject infected with HIV. In some embodiments, the method includes administering to the subject an inhibitor of CD62L shedding.

In some embodiments of the in vivo methods disclosed herein, the subject is administered an inhibitor of CD62L shedding in combination with anti-retroviral therapy (ART) or highly active anti-retroviral therapy (HAART).

Further provided is a method of inducing HIV release from infected cells by contacting the cells with an agent that induces CD62L shedding.

The foregoing and other objects, features, and advantages of the invention will become more apparent from the following detailed description, which proceeds with reference to the accompanying figures.

### BRIEF DESCRIPTION OF THE DRAWINGS

FIG. 1A: Sensorgram of serially diluted gp120 binding to immobilized soluble CD62L and a table showing solution K_{D} for CD62L binding to gp120 of a variety of HIV strains. FIG. 1B: PNGase treated gp120 resulted in reduced binding to CD62L and 2G12. FIG. 1C: ELISA binding of CD62L-Fc to immobilized gp120 in the presence of 10 mM lactose, GlcNAc (N-acetyl-D-glucosamine), sialyl-lactose, sialyl-Lewis, fucoidan, heparin, and EDTA. Sialyl-Lewis, fucoidan and heparin are known ligands of CD62L and inhibited gp120 binding (*=p≤0.05, **=p≤0.01, ***≤0.001, ****=p≤0.0001.).

FIG. 2A: Binding of gp120-QDots to immobilized soluble CD4 and CD62L in the presence and absence of CD4 and CD62L blocking antibodies. Fluorescent QDots were counted using total internal reflection fluorescence (TIRF) microscopy. FIG. 2B: gp120-QDots were added to L-selectin expressing HeLa cells and counted using TIRF microscopy (left). HeLa cells with high L-selectin expression (bright) bound more gp120-QDots those of low (dim) expression (right). FIG. 2C: A representative montage of primary CD4⁺ T cells stained for CD4, CD62L, DIC, and a merge of the CD4 and CD62L channels. Bar scale is 10 μm. FIG. 2D: Flow cytometry analysis of gp120-QDots binding to PBMC in the presence and absence of isotype, CD4, and CD62L antibodies.

FIG. 3A: Infection of activated CD8-depleted PBMCs with JRFL and SF33 pseudotyped virus produced in either 293T or 293S GnTI⁻ cells. Viral activity was detected by measuring luciferase activity. FIG. 3B: Infection of activated CD8-depleted PBMCs with JRFL and SF33 pseudotyped virus in the presence of anti-CD62L or anti-CD4 blocking antibodies or isotype control. FIG. 3C: TZM-BL and CD62L-expressing TZM-62L cells were infected with a titration of HIV-1_{BaL} and measured for luciferase activity 72 hours post infection (p.i.). FIG. 3D: Inhibition by EDTA of HIV-1_{BaL} infection in activated CD8-depleted PBMCs as measured by intracellular p24⁺ staining. FIG. 3E: Reduction by PNGase treatment of HIV-1_{BaL} infection in activated CD8-depleted PBMCs measured by real-time polymerase chain reaction (PCR) of copies of HIV-1 DNA.

FIG. 4A: Rev-CEM cells were infected with HIV-1_{BaL} in the presence of anti-CD62L or anti-CD4 blocking antibody. GFP reporter expression was measured on day 3 p.i. (left). Rev-CEM clones expressing various amounts of CD62L were infected with 40 tissue culture infectious dose 50 (TCID₅₀) of HIV-1_{BaL}. Infection was measured by real-time PCR (right). FIGS. 4B-4D: Activated CD8-depleted PBMCs were infected with serial dilutions of HIV-1_{BaL} (FIG. 4B), JRFL (FIG. 4C) and SF33 (FIG. 4D) viruses in the presence of CD62L blocking antibody or isotype controls. Infections were measured by real-time PCR (FIG. 4B) or luciferase activity (FIGS. 4C and 4D) on day 3 p.i.

FIG. 5A: Activated CD8-depleted PBMCs were infected with HIV-1_{BaL} and the expression of CD4 and CD62L was measured on day 11 p.i. on p24⁺ (solid shaded), p24⁻ (dash), and unstained cells (dotted). FIG. 5B: The expression of CD27 and CCR7 as in FIG. 5A. FIG. 5C: A representative analysis of memory CD3⁺ T cells (CD45RO⁺) gated for CD27 versus CD62L on day 6 p.i. p24⁺ (left panel) and p24⁻ (right panel). FIG. 5D: The ratio between transitional memory T cells (T_{TM}) and T_{CM} populations for paired samples on day 6 and day 11 (left). The percentage of T_{EM} cells in each paired sample on day 6 and day 11 (right). FIG. 5E: Interferon-γ expression in uninfected and day 6 p.i. activated CD8-depleted PBMCs (left panel) as well as paired p24⁺ and p24⁻ cells from the same infected sample (right panel) as measured by intracellular staining. FIG. 5F: As in FIG. 5E with additional gating for T_{CM} and T_{EM} populations.

FIG. 6A: Activated CD8-depleted PBMCs were infected with HIV-1_{BaL} with or without 5 μM BB-94 and assessed for intracellular p24 expression on Day 6 and Day 11 p.i. DMSO was used as the vehicle control. FIG. 6B: Activated CD8-depleted PBMCs were infected with JRFL- and SF33-pseudotyped virus in the presence of BB-94 or DMSO. Luciferase activity was measured at day 3 p.i. FIG. 6C: As in FIG. 6A with 100 μM BB-94 or dichloromethylenediphosphonic acid (DMDP). FIG. 6D: TZM-BL cells were co-incubated with titrating levels of infected activated CD8-depleted PBMCs in the presence of BB-94 or DMSO and measured for luciferase activity 48-60 hours p.i. FIG. 6E: HIV virion release assay. A representative of both viremic and aviremic CD4⁺ T cells activated with anti-CD3 antibody or media in the presence and absence of BB-94, DMDP or DMSO. FIG. 6F: Paired DMSO and BB-94 treatment in viral release from multiple viremic and aviremic HIV-1-infected individuals.

FIG. 7A: PNGase digestion of gp120. Lane 1—Ladder; Lane 2—Mock-treated gp120; Lane 3—PNGase-treated gp120. FIG. 7B: Binding of mock-treated and deglycosylated gp120 to CD62L as observed by surface plasmon resonance.

FIG. 8A: L-selectin glycan array results. Distribution of high affinity L-selectin ligands from 9 glycan arrays. There are a total of 13 glycan arrays performed by probing with recombinant human L-selectin in the database of Consortium for Functional Glycomics (CFG) (Hernandez et al., Blood 114, 733-741, 2009; Powlesland et al., J Biol Chem 283, 593-602, 2008). Three of the array results are labeled as inconclusive and one was done at a lower pH, thus are excluded from this analysis. The remaining 9 L-selectin glycan arrays each contain approximately 300 synthetic carbohydrates per array. They generated a total of 142 hits (so-called high affinity ligands) from 69 different carbohydrates. Among them, 20 are carbohydrate moieties from N-linked glycans, 16 are from O-linked glycans, and 33 are other types of carbohydrates. The majority of these compounds appeared as hits only in one or two of the 9 glycan arrays. The top eight compounds, however, each scored in at least four individual arrays and they count for one-third of all hits. These include the confirmed L-selectin ligands, 3′-6′-sulfo Lewis x, 3′-sulfo Lewis a, and Lewis y, as indicated 0-linked glycans. The top eight also include three compounds, as depicted here, forming part of hybrid or complex N-linked glycosylations. FIG. 8B: CD62L expression level of non-transfected TZM-BL (left peak) and transfected TZM-BL cells (right peak) as measured by flow cytometry.

FIG. 9: CD62L, CD4, CXCR4 and CCR5 expression levels of isolated Rev-CEM clones. All clones exhibited similar levels of expressions for the co-receptors. Shaded peaks are isotype controls.

FIG. 10: Day 6 post infection expression level of CD4, CD62L, CD27 and CCR7 on p24⁺ (solid shaded), p24⁻ (dash), and unstained cells (dotted). These memory markers show a decrease in CD4 and CD62L, a partial decrease in CCR7 expressions.

FIG. 11A: Gating of naïve cells is approximately 30%. FIG. 11B: Infected naïve cells show a slight decrease in CD62L expression whereas uninfected cells in the same population do not show any change in expression level of CD62L. FIG. 11C: A representative memory T cell analysis gated on CD27 and CD62L for day 11 post infection as in FIG. 5C.

FIG. 12A: Activated CD8-depleted PBMCs were treated with 100 μM BB-94 or DMSO prior to HIV-1_{BaL} infection. On day 6 p.i., the cells were stained for intracellular p24 and surface CD62L expression. The shedding of CD62L on p24⁺ PBMCs compared to p24⁻ cells was evident in DMSO treated experiment (left panel). BB94 inhibited CD62L shedding on p24⁺ PBMCs when compared to p24⁻ cells in the same infection. FIG. 12B: Activated CD8-depleted PBMCs were treated with BB-94 and DMDP at 100 μM and supernatant CD62L was measured by ELISA. DMSO was used as a vehicle control. FIG. 12C: 293T and Vero cells were infected with transitional memory T cells (VSV) in the presence of 10 μM BB-94 or DMSO. Supernatants from infected cells were used in a Vero plaque assay. Values are from duplicate measurements.

### DETAILED DESCRIPTION

### I. Abbreviations

ADAM a disintegrin and metalloproteinase
AIDS acquired immunodeficiency syndrome
ART antiretroviral therapy
CHO Chinese hamster ovary
DIC differential interference contrast
DMDP dichloromethylenediphosphonic acid
DMSO dimethyl sulfoxide
ELISA enzyme-linked immunosorbent assay
FACS fluorescence activated cell sorting
FBS fetal bovine serum
FITC fluorescein isothiocyanate
HIV human immunodeficiency virus
HRP horseradish peroxidase
IFN interferon
IL interleukin
K_{D} dissociation constant
MMP matrix metalloproteinase
PBMC peripheral blood mononuclear cell
PCR polymerase chain reaction
PE phycoerythrin
p.i. post-infection
PNGase F peptide N-glycosidase F
psi pounds per square inch
Qdot quantum dot
SPR surface plasmon resonance
TCID₅₀ tissue culture infectious dose 50
T_{CM} central memory T cells
TIRF total internal reflection fluorescence
T_{TM} transitional memory T cells
VSV transitional memory T cells

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

ADAM (a disintegrin and metalloproteinase): A family of peptidase proteins classified as sheddases because they cut off or shed extracellular portions of transmembrane proteins.

ADAM10: An ADAM family metalloproteinase that cleaves membrane proteins at the cellular surface (a sheddase). ADAM10 is known to cleave, for example, TNF-α and E-cadherin. ADAM10 is also known as CDw156 or CD156c.

ADAM17: A 70 kDa enzyme belonging to the ADAM protein family ADAM17 is a metalloproteinase responsible for the shedding of CD62L, TNF-α and other cell surface proteins involved in development, cell adhesion, migration, differentiation, and proliferation. ADAM17 is also known as tumor necrosis factor-α converting enzyme (TACE).

Administration: To provide or give a subject an agent, such as a therapeutic agent (e.g. a metalloproteinase inhibitor), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Antibody: A polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region (the regions are also known as “domains”). References to “V_{H}” or “V_{H}” refer to the variable region of an immunoglobulin heavy chain, including that of an Fv, scFv, dsFv or Fab. References to “V_{L}” or “V_{L}” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a murine antibody.

A “humanized” immunoglobulin is an immunoglobulin including a human framework region and one or more complementarity determining regions (CDRs) from a non-human (for example a mouse, rat, or synthetic) immunoglobulin. The non-human immunoglobulin providing the CDRs is termed a “donor,” and the human immunoglobulin providing the framework is termed an “acceptor.” Generally, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

Anti-retroviral agent: An agent that specifically inhibits a retrovirus from replicating or infecting cells. Non-limiting examples of antiretroviral drugs include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir) and maturation inhibitors (e.g., alpha interferon, bevirimat and vivecon).

Anti-retroviral therapy (ART): A therapeutic treatment for HIV infection involving administration of at least one anti-retroviral agent (e.g., one, two, three or four anti-retroviral agents) to an HIV infected individual during a course of treatment. Non-limiting examples of antiretroviral agents include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir) and maturation inhibitors (e.g., alpha interferon, bevirimat and vivecon). One example of an ART regimen includes treatment with a combination of tenofovir, emtricitabine and efavirenz. In some examples, ART includes Highly Active Anti-Retroviral Therapy (HAART).

Batimastat: A synthetic matrix metalloproteinase inhibitor that has been used as an anti-cancer agent. Batimastat is also known as BB-94.

CD62L: A cell adhesion molecule found on lymphocytes and the preimplantation embryo. CD62L belongs to the selectin family of proteins, which recognize sialylated carbohydrate groups. Shedding of CD62L on activated T cells is primarily mediated by ADAM17. CD62L is also known as L-selectin.

Contacting: Placement in direct physical association; includes both in solid and liquid form.

Human immunodeficiency virus (HIV): A retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by HIV, as determined by antibody or western blot studies. Laboratory findings associated with this disease include a progressive decline in T cells. HIV includes HIV type 1 (HIV-1) and HIV type 2 (HIV-2). Related viruses that are used as animal models include simian immunodeficiency virus (SIV), and feline immunodeficiency virus (FIV). Treatment of HIV-1 with HAART has been effective in reducing the viral burden and ameliorating the effects of HIV-1 infection in infected individuals.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Lentivirus: A genus of retroviruses characterized by a long incubation period and the ability to infect non-dividing cells. Lentiviruses typically cause chronic, progressive, and often fatal disease in humans and other animals. Examples of lentiviruses include HIV, SIV, FIV and EIAV.

Matrix metalloproteinase (MMP): A family of zinc-dependent neutral endopeptidases that play a role in degradation and remodeling of the ECM. MMPs are also known to be involved in the cleavage of cell surface receptors, the release of apoptotic ligands (such as the FAS ligand), chemokine activation and inactivation. MMPs are also thought to play a major role in cell proliferation, migration (adhesion/dispersion), differentiation, angiogenesis, apoptosis and host defense. At least 22 mammalian MMPs have been identified and are categorized based on structure and substrate specificity. The designated subgroups of MMPs include collagenases (MMP-1, MMP-8, MMP-13), stromelysins (MMP-3, MMP-10, MMP-11, MMP-12), matrilysins (MMP-7, MMP-26), gelatinases (MMP-2, MMP-9), membrane-type MMPs (MMP14, MMP-15, MMP-16, MMP-17, MMP-24), and other uncategorized MMPs (MMP-19, MMP-20, MMP-23, MMP-25, MMP-27, MMP-28) (Johansson et al., Cell. Mol. Life Sci. 57:5-15, 2000; Egeblad and Werb, Nat. Rev. Cancer 2:161-174, 2002).

Matrix metalloproteinase (MMP) inhibitor: A molecule that inhibits the activity or function of a MMP. MMP inhibitors include, but are not limited to, small molecules, antibodies, nucleic acid molecules, peptide inhibitors and chelating compounds. Exemplary MMP inhibitors include Batimastat (BB-49), Marimastat, AG3340, BAY 12-9566 and CGS27023A (Rothenberg et al., The Oncologist 3(4):271-274, 1998). Other MMP inhibitors are well known in the art (see, for example, PCT Publication No. WO 00/38718). Exemplary MMP inhibitors are disclosed herein (see section V).

Metalloproteinase: An enzyme whose catalytic mechanism involves a metal. Most metalloproteinases require zinc, but some use cobalt. Metalloproteinases include exopeptidases and endopeptidases. Endopeptidases include, for example, matrix metalloproteinases and ADAM proteins.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Retroviruses: Enveloped viruses that replicate in a host cell through the process of reverse transcription. Retroviruses are positive sense, single-stranded RNA viruses with a spherical particle of about 80 to about 120 nm in diameter. Retrovirus particles contain two copies of the positive strand RNA genome. The retrovirus genome includes three primary genes coding for the viral proteins—gag-pol-env, and two regulatory genes—tat and rev. Retroviruses also have additional accessory proteins, depending on the particular virus. For example, the HIV genome includes the vif, vpr, vpu and nef genes.

Sheddase: A membrane-bound enzyme that cleaves extracellular portions of transmembrane proteins, releasing the soluble ectodomains from the cell surface. Many sheddases are members of the ADAM or aspartic protease (BACE) protein families.

Shedding: Cleavage and release of the ectodomain of a transmembrane protein. “CD62L shedding” refers to cleavage of the extracellular portion (ectodomain) of CD62L from the surface of a cell.

Small molecule: A molecule, typically with a molecular weight less than about 1000 Daltons, or in some embodiments, less than about 500 Daltons, wherein the molecule is capable of modulating, to some measurable extent, an activity of a target molecule.

Subject: Living multi-cellular vertebrate organisms, a category that includes human and non-human mammals.

Synthetic: Produced by artificial means in a laboratory, for example a synthetic nucleic acid can be chemically synthesized in a laboratory.

Therapeutically effective amount: A quantity of a specified pharmaceutical or therapeutic agent (e.g. a recombinant vector) sufficient to achieve a desired effect in a subject, or in a cell, being treated with the agent. The effective amount of the agent will be dependent on several factors, including, but not limited to the subject or cells being treated, and the manner of administration of the therapeutic composition.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Introduction

Previous envelope glycan mutation experiments have shown that while many gp120 glycans modulated viral sensitivity to neutralization antibodies, some also affected the virulence of the virus (Johnson et al., J Virol 75, 11426-11436, 2001; Kolchinsky et al., J Virol 75, 3435-3443, 2001). The role of envelope-associated glycans is thought to provide a ‘glycan shield’ to evade the host adaptive immune response (Quinones-Kochs et al., J Virol 76, 4199-4211, 2002). However, studies have shown that HIV-neutralizing antibodies can also recognize glycans as part of their epitopes (Trkola et al., J. Virol. 70, 1100-1108, 1996; McLellan et al., Nature 480, 336-343, 2011). In addition, gp120 associated glycans have been shown to bind lectin receptors, such as Siglecs, to facilitate viral adhesion to macrophages (Zou et al., PLoS One 6(9), e24559, 2011). The trimeric structure of HIV-1 gp140 shows glycans forming an outer layer canopy of the envelope protein (Harris et al., Proc Natl Acad Sci USA 108, 11440-11445, 2011). The inventors investigated the possibility that viral envelope glycans may recognize lectin receptors on T cells. One such lectin receptor that is expressed on CD4⁺ T cells is L-selectin (CD62L), a marker for both central memory and naïve T cells. It is a C-type lectin receptor that recognizes sulfated sialyl-Lewis X on P-selectin glycoprotein ligand-1 (PSGL-1) and other mucin-like proteoglycans on endothelial cells. CD62L enables T cells to undergo fast-rolling adhesion and homing to various tissues (von Andrian et al., Microcirculation 3, 287-300, 1996).

It is disclosed herein that L-selectin/CD62L serves as a viral adhesion receptor on CD4⁺ T cells. HIV-1 envelope glycans recognize CD62L on CD4⁺ T cells, resulting in preferential infection of CD62L⁺ central memory T cells. It is further disclosed herein that HIV-infection activates shedding of CD62L and downregulation of CCR7. Inhibition of CD62L shedding dramatically reduced HIV-1_{BaL} infection and inhibited viral release in both viremic and aviremic patient CD4⁺ T cells, indicating that CD62L expressing T cells form a potential HIV reservoir.

### IV. Overview of Several Embodiments

It is disclosed herein that HIV envelope glycans bind CD62L (L-selectin) on central memory T cells. It is further disclosed that HIV infection induces shedding of CD62L and this shedding is required for the efficient release of HIV from infected cells. Thus, methods of inhibiting HIV release from infected cells using inhibitors of CD62L sheddase are provided. Methods of treating HIV infection with a CD62L sheddase, such as in combination with antiretroviral therapy, are also provided.

Provided herein are methods of inhibiting HIV release from an infected cell by contacting the cell with an inhibitor of CD62L shedding. In some embodiments, the inhibitor is a metalloproteinase inhibitor, such as an inhibitor that reduces or blocks the sheddase activity of a metalloproteinase. In some examples, the metalloproteinase inhibitors is a matrix metalloproteinase (MMP) inhibitor. In other examples, the metalloproteinase inhibitor is a disintegrin and metalloproteinase (ADAM) family inhibitor, such as an inhibitor of ADAM17, an inhibitor of ADAM10, or an inhibitor of both ADAM17 and ADAM10.

The inhibitor can be any type of molecule that inhibits the sheddase activity of a protein that mediates CD62L shedding, such as a molecule that inhibits the sheddase activity of a MMP or an ADAM. In some embodiments, the CD62L sheddase inhibitor is a small molecule. In specific non-limiting examples, the small molecule is a MMP inhibitor, such as marimastat (BB-2516), batimastat (BB-94), prinomastat (AG3340), tanomastat (BAY 12-9566) or BB1101. Other exemplary MMP inhibitors are known in the art, some of which are listed in section V below.

In some embodiments, the CD62L sheddase inhibitor specifically inhibits an ADAM protein, such as ADAM17 and/or ADAM10. In some examples, the ADAM17 or ADAM10 inhibitor is INCB007839, INCB3619, BB3103, BMS-561392, BMS-566394, DPC-A38088, DPH-067517, GW280264X, GW4459, IK682, IM491, INCB4298, INCB7839, R-618 or TMI-2. Other exemplary ADAM17 and/or ADAM10 inhibitors are known in the art and/or are listed in section V below.

In some embodiments, the inhibitor of CD62L shedding is an antibody. In some examples, the antibody is a polyclonal antibody, or an antigen-binding fragment thereof. In other examples, the antibody is a monoclonal antibody, or an antigen-binding fragment thereof.

In some embodiments, the methods of inhibiting HIV release are in vitro or ex vivo methods in which cells infected with HIV are contacted with the inhibitor of CD62L shedding. In some examples, the cells are T lymphocytes, such as memory T cells. In particular non-limiting examples, the T cells are central memory CD4⁺ T cells.

In other embodiments, the methods of inhibiting HIV release are in vivo methods in which contacting the cell with an inhibitor of CD62L shedding includes administering the inhibitor to a subject infected with HIV.

Further provided herein are methods of treating a subject infected with HIV. In some embodiments, the method includes administering to the subject an inhibitor of CD62L shedding. In some embodiments, the inhibitor is a metalloproteinase inhibitor, such as an inhibitor that reduces or blocks the sheddase activity of a metalloproteinase. In some examples, the inhibitor is a MMP inhibitor. In other examples, the metalloproteinase inhibitor is an ADAM inhibitor, such as an inhibitor of ADAM17, an inhibitor of ADAM10, or an inhibitor of both ADAM17 and ADAM10.

The inhibitor used in the disclosed methods of treating a subject with HIV can be any type of molecule that inhibits the sheddase activity of a protein that mediates CD62L shedding, such as a molecule that inhibits the sheddase activity of a MMP or an ADAM. In some embodiments, the CD62L sheddase inhibitor is a small molecule. In specific non-limiting examples, the small molecule is a MMP inhibitor, such as marimastat (BB-2516), batimastat (BB-94), prinomastat (AG3340), tanomastat (BAY 12-9566) or BB1101. Other exemplary MMP inhibitors are known in the art, some of which are listed in section V below.

In some embodiments of the treatment methods, the CD62L sheddase inhibitor specifically inhibits ADAM17 and/or ADAM10. In some examples, the ADAM17 or ADAM10 inhibitor is INCB007839. INCB3619, BB3103, BMS-561392, BMS-566394, DPC-A38088, DPH-067517, GW280264X, GW4459, IK682, IM491, INCB4298, INCB7839, R-618 or TMI-2. Other exemplary ADAM17 and/or ADAM10 inhibitors are known in the art and/or are listed in section V below.

In some embodiments of the methods of treating HIV, the inhibitor of CD62L shedding is an antibody. In some examples, the antibody is a polyclonal antibody, or an antigen-binding fragment thereof. In other examples, the antibody is a monoclonal antibody, or an antigen-binding fragment thereof.

In some embodiments, the methods further include treatment of a subject with another type of therapy or therapeutic agent. In some examples, the subject is further administered anti-retroviral therapy (ART) or highly active anti-retroviral therapy (HAART). ART can include, for example, administration of at least one anti-retroviral agent to an HIV infected individual during a course of treatment. In some examples, the subject is administered two, three, four, five, six, seven or eight anti-retroviral agents. Non-limiting examples of antiretroviral agents include entry/fusion inhibitors (such as enfuvirtide or maraviroc), CCR5 receptor antagonists (such as aplaviroc, vicriviroc or maraviroc), reverse transcriptase inhibitors (such as lamivudine, zidovudine, abacavir, tenofovir, emtricitabine or efavirenz), protease inhibitors (such as lopivar, ritonavir, raltegravir, darunavir or atazanavir), maturation inhibitors (such as alpha interferon, bevirimat or vivecon), or integrase inhibitors (such as raltegravir, elvitegravir, dolutegravir or MK-2048). One non-limiting example of an ART regimen includes treatment with a combination of tenofovir, emtricitabine and efavirenz. HAART encompasses any highly aggressive treatment regimens for patients infected with HIV. In some examples, HAART includes three or more (such as four, five, six, seven, or eight or more) different anti-retroviral drugs, such as, but not limited to, two reverse transcriptase inhibitors and a protease inhibitor.

In some embodiments, a patient infected with HIV is administered a CD62L shedding inhibitor along with the combination of efavirenz, tenofovir and emtricitabine; ritonavir-boosted atazanavir, tenofovir and emtricitabine; ritonavir-boosted darunavir, tenofovir and emtricitabine; or altegravir, tenofovir and emtricitabine.

Further provided herein are compositions comprising an inhibitor of CD62L shedding and an anti-retroviral agent. In some examples, the composition comprises an inhibitor of CD62L shedding disclosed herein (such as a metalloproteinase inhibitor) and an entry/fusion inhibitor (such as enfuvirtide or maraviroc), a CCR5 receptor antagonist (such as aplaviroc, vicriviroc or maraviroc), a reverse transcriptase inhibitor (such as lamivudine, zidovudine, abacavir, tenofovir, emtricitabine or efavirenz), a protease inhibitor (such as lopivar, ritonavir, raltegravir, darunavir or atazanavir), a maturation inhibitor (such as alpha interferon, bevirimat or vivecon), or an integrase inhibitor (such as raltegravir, elvitegravir, dolutegravir or MK-2048). One non-limiting example, the composition comprises an inhibitor of CD62L and one or more of tenofovir, emtricitabine and efavirenz.

Also provided herein is a method of inducing HIV release from infected cells by contacting the cells with an agent that induces CD62L shedding. In some embodiments, the infected cells are latent HIV reservoirs. In some examples, the infected cells are central memory T cells. In some embodiments, the method is an in vitro or ex vivo method in which CD62L-expressing cells are contacted with the agent. In other embodiments, the method is an in vivo method in which a subject with HIV is administered an agent that induces CD62L shedding. In some embodiments, the method further includes administering to the subject ART or HAART.

### V. Metalloproteinase Inhibitors

Any inhibitor, such as a metalloproteinase inhibitor, that is capable of inhibiting CD62L shedding is contemplated for use in the methods disclosed herein. In some embodiments, the metalloproteinase inhibitor is a matrix metalloproteinase (MMP) inhibitor. A variety of MMP inhibitors are known in the art. For example, the following patent and scientific publications provide descriptions of specific MMP inhibitors, classes of MMP inhibitors, and methods of making and testing MMP inhibitors: U.S. Pat. Nos. 5,831,004; 6,265,432; 6,307,101; 6,339,160; 6,350,885; and 6,133,304 (each of which is incorporated herein by reference); and Kleiner and Stetler-Stevenson, Canc. Chemother. Pharmacol. 43 Suppl:S42-51, 1999. Exemplary MMP inhibitors include Marimastat (BB-2516), Batimastat (BB-94), Prinomastat (AG3340), Tanomastat (BAY 12-9566) and BB1101 (Barlaam et al., J Med Chem 42(23):4890-4908, 1999).

In some embodiments disclosed herein, the metalloproteinase inhibitor is an ADAM protein inhibitor, such as an inhibitor of ADAM17 and/or ADAM10. In some examples, the ADAM17 and/or ADAM10 inhibitor is a small molecule inhibitor specific for ADAM17 and/or ADAM10.

The following table provides a non-limiting list of small molecule ADAM inhibitors, including inhibitors that target ADAM17 and/or ADAM10:

| Inhibitor | Target(s) | Reference |
|---|---|---|
| BB3103 | ADAM17 | Hurtado et al., J Cereb Blood Flow Metab 22(5): 576-585, |
| | ADAM10 | 2002 |
| BMS-561392 | ADAM17 | Grootveld and McDermott, Curr Opin Investig Drugs |
| | | 4(5): 598-602, 2003 |
| BMS-566394 | ADAM17 | Moss et al., Nat Clin Pract Rheumatol 4(6): 300-309, 2008 |
| CH-138 | ADAM17 | Moss et al., Nat Clin Pract Rheumatol 4(6): 300-309, 2008 |
| | MMPs | |
| DPC-A38088 | ADAM17 | Moss et al., Nat Clin Pract Rheumatol 4(6): 300-309, 2008 |
| DPH-067517 | ADAM17 | 207 |
| GI-5402 | ADAM17 | Dekkers et al., Blood 94(7): 2252-2258, 1999 |
| | MMPs | |
| GM6001 | ADAM17 | Mirastschijski et al., Eur Surg Res 37(1): 68-75, 2005 |
| | MMPs | |
| GW-3333 | ADAM17 | Levin et al., Bioorg Med Chem Lett 11(2): 239-242, 2001 |
| | MMPs | |
| GW280264X | ADAM17 | Hundhausen et al., Blood 102(4): 1186-1195, 2003 |
| | ADAM10 | |
| GW4459 | ADAM17 | Rabinowitz et al., J Med Chem 44(24): 4252-4267, 2001 |
| IK682 | ADAM17 | Niu et al., Arch Biochem Biophys 451(1): 43-50, 2006 |
| IM491 | ADAM17 | Xue et al., Bioorg Med Chem Lett 13(24): 4299-4304, 2003 |
| INCB3619 | ADAM17 | Fridman et al., Clin Cancer Res 13(6): 1892-1902, 2007 |
| | ADAM10, | |
| | MMPs | |
| INCB4298 | ADAM17 | Zhou et al., Cancer Cell 10(1): 39-50, 2006 |
| INCB7839 | ADAM17 | Morimoto et al., Life Sci 61(8): 795-803, 1997 |
| | ADAM10 | |
| KB-R7785 | ADAM17 | Barlaam et al., J Med Chem 42(23): 4890-4908, 1999 |
| | ADAM12 | |
| | MMPs | |
| PKF242-484 | ADAM17 | Trifilieff et al., Br J Pharmacol 135(7): 1655-1664, 2002 |
| | MMPs | |
| PKF241-466 | ADAM17 | Trifilieff et al., Br J Pharmacol 135(7): 1655-1664, 2002 |
| | MMPs | |
| R-618 | ADAM17 | Moss et al., Nat Clin Pract Rheumatol 4(6): 300-309, 2008 |
| TAPI-1 | ADAMs, | Mohler et al., Nature 370: 218-220, 1994 |
| | MMPs | |
| TAPI-2 | ADAMs, | Arribas et al., J Biol Chem 271(19): 11376-11382, 1996 |
| | MMPs | |
| TMI-005 | ADAM17 | Thabet et al., Curr Opin Investig Drug 7(11): 1014-1019, |
| | MMPs | 2006 |
| TMI-1 | ADAM17 | Zhang et al., Int Immunopharmacol 4(14): 1845-1857, 2004 |
| | MMPs | |
| TMI-2 | ADAM17 | Zhang et al., J Pharmacol Exp Ther 309(1): 348-355, 2004 |
| W-3646 | ADAM17 | Moss et al., Nat Clin Pract Rheumatol 4(6): 300-309, 2008 |
| WTACE2 | ADAM17 | Dell et al., Kidney Int 60(4): 1240-1248, 2001 |
| XL784 | ADAM17 | McCarthy, Chem Biol 12(4): 407-408, 2005 |
| | ADAM10 | |
| | MMPs | |

Additional small molecule inhibitors of ADAM17 have been previously described, such as in the following references: Minond et al., J Biol Chem 287(43):36473-36487, 2012; Arribas and Esselens, Curr Pharm Des 15(20):2319-2335, 2009; Nuti et al., J Med Chem 56(20):8089-8103, 2013; Bandarage et al., Bioorg Med Chem Lett 18(1):44-48, 2008; Condon et al., Bioorg Med Chem Lett 17(1):34-39, 2007; Duan et al., Bioorg Med Chem Lett 13(12):2035-2040, 2003; Tsukida et al., Bioorg Med Chem Lett 14(6):156-1572, 2004; Levin et al., Bioorg Med Chem Lett 12(8):1199-1202, 2002; Xue et al., J Med Chem 44(21):3351-3354, 2001; Letavic et al., Bioorg Med Chem Lett 12(10):1387-1390, 2002; Levin et al., Bioorg Med Chem Lett 13(16):2799-2803, 2003; Duan et al., J Med Chem 45(23):4954-4957, 2002; Sawa et al., Bioorg Med Chem Lett 13(12):2021-2024, 2003; Cherney et al., Bioorg Med Chem Lett 16(4):1028-1031, 2006; Holms et al., Bioorg Med Chem Lett 11(22):2907-2910, 2001; Kamei et al., Bioorg Med Chem Lett 14(11):2897-2900, 2004; Cherney et al., J Med Chem 46(10):1811-1823, 2003; Venkatesan et al., J Med Chem 47(25):6255-6269, 2004; Blacker et al., J Neurochem 83(6):1349-1357, 2002; and U.S. Application Publication Nos. 2007/0280943, 2004/0259896, 2005/0250789 and 2005/0113344, which are herein incorporated by reference.

In some embodiments, the CD62L sheddase inhibitor is a sulfonic acid or phosphinic acid derivative, such as a sulfonic acid or phosphinic acid derivative that inhibits ADAM17. Sulfonic acid or phosphinic acid derivatives include sulfonamides, sulfonamide hydroxamic acids, phosphinic acid amide hydroxamic acids (for example those described in U.S. Application Publication No. 2009/0292007; PCT Publication Nos. WO 98/16503, WO 98/16506, WO 98/16514, WO 98/08853, WO 98/03166, WO 97/18194 and WO 98/16520, which are herein incorporated by reference; Mac Pherson et al., J Med Chem 40:2525, 1997; Tamura et al., J Med Chem 41:690, 1998; Levin et al., Bioorg Med Chem Lett 8:2657, 1998; and Pikul et al., J Med Chem 41:3568, 1998).

In some embodiments, the CD62L sheddase inhibitor is a cyclic peptide that inhibits TACE/ADAM17 (or other metalloproteinases), such as the peptides described in U.S. Application Publication No. 2015/0080319, which is herein incorporated by reference.

In some examples, the CD62L sheddase inhibitor is INCB007839, which is an orally bioavailable inhibitor of the ADAM family of proteins. Sheddase inhibitor INCB007839 represses the metalloproteinase sheddase activities of ADAM10 and ADAM17.

In some examples, the CD62L sheddase inhibitor is INCB3619, which is an orally bioavailable small-molecule inhibitor of a subset of ADAM proteases (Fridman et al., Clin Cancer Res 13(6): 1892-1902, 2007).

In some embodiments, the metalloproteinase inhibitor is an antibody, such as a monoclonal antibody (or antigen-binding fragment thereof), that specifically binds and inhibits the sheddase activity of a metalloproteinase, such as an MMP or ADAM. In some examples, the antibody specifically binds and inhibits ADAM17 or ADAM10 (see, for example, Caiazza et al., Br J Cancer 112:1895-1903, 2015). In other examples, the antibody or antigen-binding fragment is a monoclonal or polyclonal antibody produced using any technique known in the art (see section VI below).

### VI. Antibodies Specific for CD62L Sheddase

In some embodiments disclosed herein, the inhibitor of CD62L shedding is an antibody, or antigen-binding fragment of an antibody, that specifically binds and inhibits the sheddase activity of the target protein. In some embodiments, the CD62L sheddase (the target protein) is a metalloproteinase. In particular examples, the metalloproteinase is a MMP. In other particular examples, the metalloproteinase is ADAM17 or ADAM10.

In some embodiments, the antibody is a polyclonal antibody. In other embodiments, the antibody is a monoclonal antibody, or an antigen-binding fragment thereof.

Polyclonal antibodies, antibodies which consist essentially of pooled monoclonal antibodies with different epitopic specificities, as well as distinct monoclonal antibody preparations are included. The preparation of polyclonal antibodies is well known to those skilled in the art (see, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992).

The preparation of monoclonal antibodies likewise is conventional (see, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992).

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, such as syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from a subhuman primate antibody. General techniques for raising therapeutically useful antibodies in baboons can be found, for example, in PCT Publication No. WO 91/11465; and Losman et al., Int. J. Cancer 46:310, 1990.

Alternatively, an antibody that specifically binds a target protein can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from Stratagene Cloning Systems (La Jolla, Calif.).

Monoclonal antibodies can be derived from antibody phage libraries, such as scFv phage libraries. In some embodiments, the phage library is a fully human scFv phage library (see, e.g. Li et al., Protein Eng Des Sel 28(10)307-316, 2015; Hammers and Stanley, J Invest Dermatol 134(2):e17, 2014).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)2, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody, defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Natl. Acad. Sci. U.S.A. 69:2659, 1972). Alternatively, the variable chains can be linked by an intermolecular disulfide bond or cross-linked by chemicals such as glutaraldehyde (see, for example, Sandhu, Crit. Rev. Biotech. 12:437, 1992). Preferably, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using an intact polypeptide or fragments containing small peptides of interest as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly used carriers which are chemically coupled to the peptide include keyhole limpet hemocyanin, thyroglobulin, bovine serum albumin, and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see, for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first monoclonal antibody.

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab−Ag]/[Ab][Ag] where [Ab] is the concentration at equilibrium of the antibody, [Ag] is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

### VII. Inducing CD62L Shedding to Eliminate HIV Reservoirs

The present disclosure also contemplates the use of agents that induce CD62L (L-selectin) shedding as a means to release latent HIV from cell reservoirs. Despite the success of ART in controlling HIV in infected individuals, treatment is less effective at eliminating HIV viral reservoirs. The nature of HIV reservoirs and the factors controlling their size and release are a major research focus for achieving a cure for HIV/AIDS. The data disclosed herein indicate that CD62L expressing T cells form a potential HIV reservoir.

Provided herein is a method of inducing HIV release from cells, such as cell reservoirs, comprising contacting the cells or cell reservoirs with an agent that induces CD62L shedding. In some embodiments, the method is an in vitro or ex vivo method in which CD62L-expressing cells (such as central memory T cells) are contacted with the agent. In other embodiments, the method is an in vivo method in which a subject with HIV is administered an agent that induces CD62L shedding. In some embodiments, the method further includes administering to the subject ART or HAART.

Agents that induce CD62L shedding are known in the art (see, for example, U.S. Pat. No. 6,949,665, which is herein incorporated by reference). In some embodiments, the agent is an anti-thiol agent.

The following examples are provided to illustrate certain particular features and/or embodiments. These examples should not be construed to limit the disclosure to the particular features or embodiments described.

### EXAMPLES

The mechanisms that dictate preferential infection of central memory CD4⁺ T cells by HIV-1, as well as the factors contributing to the persistence of viral reservoirs, are not well understood but are central to controlling HIV-1 infections. In the studies described in the examples, L-selectin/CD62L was identified as a viral adhesion receptor on CD4⁺ T cells. HIV-1 envelope glycans recognized CD62L on CD4 T cells resulting in preferential infection of CD62L⁺ central memory T cells. HIV-infection activated shedding of CD62L and downregulation of CCR7, explaining the preferential loss of central memory CD4 T cells in HIV patients. The infected effector memory CD4 T cells were, however, competent in cytokine production, suggesting that the appearance of dysfunctional effector memory CD4⁺ T cells in HIV patients is related to misidentifying infected central memory as effector memory T cells. Inhibition of CD62L shedding dramatically reduced HIV-1_{BaL} infection and inhibited viral release in both viremic and aviremic patient CD4⁺ T cells, indicating that CD62L expressing T cells form a potential HIV reservoir. The requirement of CD62L shedding in HIV viral release opens a new avenue of antiviral treatment.

### Example 1: Materials and Methods

This example describes the experimental procedures for the studies described in Example 2.

### Reagents

Unless otherwise specified, all reagents and chemicals were purchased from Sigma-Aldrich Co. (St. Louis, Mo.). Recombinant protein was purchased from R&D Systems, Inc. (Minneapolis, Minn.). Other recombinant CD62L was prepared from stably transfected Chinese hamster ovary (CHO) cells using an expression system described previously (Zou and Sun, Protein Expr Purif 37, 265-272, 2004). Blocking antibody against CD62L (DREG-56) was harvested from hybridoma cells in serum-free media from Invitrogen (Carlsbad, Calif.) and purified using a Protein A column or purchased from eBioscience (San Diego, Calif.). Unlabeled mouse anti-human CD4 monoclonal antibody (RPA-T4), CD3 (OKT3) and CD28 were obtained from eBioscience (San Diego, Calif.). Fluorescently labeled antibodies for flow cytometry against CD14, CXCR4, CCR5, CD8, CD4, CD3, CD62L, CD27, CD45RO, CCR7, interferon (IFN)γ and their isotype controls (IgG1, IgG2A, IgG2B) were obtained from BD Biosciences (San Jose, Calif.), BioLegend (San Diego, Calif.) or eBioscience (San Diego, Calif.). Alexa-647 labeled antibodies used for confocal microscopy were obtained from BioLegend (San Diego, Calif.). HIV-1 core antigen antibody (KC57-FITC) for intracellular p24 staining was purchased from Beckman Coulter, Inc. (Miami, Fla.). Interleukin 2 (IL-2) was obtained from Peprotech Inc. (Rocky Hill, N.J.). Polyacrylamide (PAA)-conjugated model carbohydrates were obtained from Glycotech, Inc. (Rockville, Md.). All other carbohydrates were purchased from Carbosynth Ltd. (Compton, Berkshire, UK). Recombinant gp120 proteins were expressed in CHO or 293T cells in monomeric forms as previously described (Cicala et al., Proc Natl Acad Sci USA 103, 3746-3751, 2006). The Luciferase Assay System was purchased from Promega Corporation (Madison, Wis.). HIV-1 p24 ELISA kit was obtained from PerkinElmer Life Sciences, Inc. (Waltham, Mass.). FICOLL-PAQUE™ was purchased from GE Healthcare Bio-Sciences (Pittsburgh, Pa.). For fluorescence activated cell sorting (FACS) analysis, recombinant gp120 proteins were labeled with biotin using a biotinylation kit from Pierce Biotechnology (Rockford, Ill.). RPMI, penicillin/streptomycin, fetal bovine serum, HEPES, and Versene for peripheral blood mononuclear cell (PBMC) experiments were purchased from Invitrogen Corporation (Carlsbad, Calif.). The metalloproteinase inhibitor BB94 (Batimastat) was purchased from Santa Cruz Biotechnology.

### Activation and Expansion of Peripheral Blood Mononuclear Cells

PBMCs were isolated from randomly selected non-identified healthy donors by FICOLL-PAQUE™ gradient. The isolated PBMCs were plated at 3×10⁶/mL in 12-well plates with RPMI supplemented with 10% fetal bovine serum (FBS), 1% penicillin/streptomycin and 20 U/mL IL-2. CD8+ cell depletion (CD8− PBMCs) was completed using the StemCell (Vancouver, BC, Canada) EASYSEP™ Human CD8 Positive Selection Kit prior to infection. Total cell counts and viability determinations were assessed with the Guava Personal Cell Analysis System (Guava Technologies) or the Luna FL Dual Fluorescence Cell Counter (Logos Biosystems); all assays were performed with a cellular viability greater than 90%.

### Stable Selectin Transfected HeLa Cells

HeLa cells were cultured in DMEM/F12 medium supplemented with 5% FBS. Neomycin-resistant vectors from GeneCopoeia (Rockville, Md.) containing coding regions for human CD62L were transfected into HeLa cells using LIPOFECTAMINE™ 2000 from Invitrogen (Carlsbad, Calif.). G418-resistant cells were expanded and used as a mixed population for TIRF microscopy. High and low selectin-expressing HeLa cells were sorted on a BD FACS Aria II using 100 μm nozzle at a pressure of 20 psi. Cells were labeled with Alexa-647 conjugated antibodies against L-selectin. The sorted populations were expanded in the same growth media. To reduce cleavage of selectins from the cell surface, sorted and unsorted transfected cells were grown in NUNC™ UPCELL™ 6-well plates from Thermo Scientific (Waltham, Mass.) and released after incubating at room temperature in versene and gentle pipetting.

### Rev-CEM Cloning

Rev-CEM cells were obtained from the NIH AIDS Reagent Program. Single clones were selected based on CD62L cell surface expression. Three different groups of rev-CEM clones were obtained with high, medium and low expression of CD62L based on FACS analysis. The expression level of CD62L on Rev-CEM cells was stable for at least two weeks before the cells were used for HIV virus infection. Cells were grown and maintained in PBMC media without IL-2.

### Preparation of Pseudotyped HIV

The HIV vector pNL4-3.Luc.R-E-, which contains the firefly luciferase gene inserted into the NL4-3 HIV nef gene and frameshift mutations to render it E-, was used to generate all pseudotyped viruses (He et al., J Virol 69, 6705-6711, 1995; Connor et al., Virology 206, 935-944, 1995).

In brief, the expression vectors for pNL4-3.Luc.R-E-, the amphotropic envelope pHEF-VSVG, and the R5-tropic HIV JRFL envelope were obtained through the NIH AIDS Research and Reference Reagent Program. Expression vectors for the X4-tropic SF33 HIV-1 envelope have been described (Cho et al., J Virol 72, 2509-2515, 1998). Recombinant HIV luciferase viruses were generated by co-transfecting 293T cells with 5 μg of the NL4-3 backbone and either 5 μg of the HIV envelopes or 1.5 μg of the VSV envelope, as previously described (Moir et al., J Exp Med 192, 637-646, 2000). Virus collected in the culture supernatant were quantified by HIV p24 ELISA and adjusted to 1 mg/mL p24. Pseudotyped virus deficient for complex carbohydrates were generated as above, but transfected into HEK 293S GNTI⁻ cells obtained from American Type Culture Collection (Manassas, Va.).

### Replication Competent Virus

The R5-tropic Ba-L strain of HIV-1 virus (HIV-1_{BaL}), propagated using primary human macrophages, was purchased from Advanced Biotechnologies Inc. (Columbia, Md.). Aliquots of 50 μL were frozen and saved for future use. Initial virus stock was grown from a frozen aliquot in CD8− PBMCs. Day 6 supernatant was harvested and 200 μL aliquots were frozen. A sample from the supernatant was titrated in CD8− PBMCs to determine the optimal dilution for infection. TCID₅₀ was measured on CD8− PBMCs by p24 ELISA from PerkinElmer Life Sciences, Inc. (Waltham, Mass.) per the manufacturer's instructions.

Soluble gp120 and CD62L ELISA

Fifty ng of gp120 proteins were immobilized in individual wells of a 96-well plate for 1 hour at room temperature in coating buffer (10 mM Tris [pH7.5] and 2 mM CaCl₂), blocked for 1 hour using blocking buffer (10 mM Tris [pH7.5], 0.1% Tween20), and washed three times with the same buffer. Fc-CD62L (25 ng) was added to each well in the presence or absence of various inhibitors (10 mM EDTA or 10 mg/ml carbohydrates) together with a goat anti-human IgG-horseradish peroxidase (HRP) secondary antibody for 1 hour at room temperature. The plate was washed five times and readout was colorimetric using a TMB substrate and analyzed on a SPECTRAMAX™ Plus 384 spectrophotometer (Molecular Devices).

### Surface Plasmon Resonance

Surface plasmon resonance measurements were performed using a BIACORE™ 3000 instrument (GE Healthcare). Recombinant CD62L-Fc, CD62L (R and D), or CD62L expressed in CHO cells was immobilized onto carboxymethylated dextran (CMS) surface-based sensor chips by N-hydrosuccinimide/1-ethyl-3(-3-dimethylaminopropyl) carbodiimide hydrochloride (NHS/EDC) crosslinking in sodium acetate buffer, pH4.5 or 5.0 Immobilization level was 400-900 RU for dilution experiments and PNGase comparison. The 2G12 antibody was immobilized (RU 480) in sodium acetate buffer pH 5.0. Flow cell 1 was mock immobilized as a blank in all cases. Binding assays were run in HBS-P+Ca (10 mM HEPES pH 7.2, 150 mM NaCl, 0.005% P20, and 0.5-2 mM CaCl₂). Recombinant gp120 proteins with varying concentrations between 10-500 nM, in HBS-P+Ca buffer, were injected over immobilized receptors at a flow rate of 20 mL/min. For carbohydrate removal gp120-205F was treated with 2 units PNGase per μg of gp120 in 50 mM Na phosphate at pH 7.5 overnight at 37° C. The dissociation constants (K_{D}) were determined from kinetic curve fitting using the BIAevaluation software (GE Healthcare).

### Single-Round Infection Assay

Stimulated CD8− PBMCs were resuspended at 2×10⁶/mL in culture media. Aliquots of 200 (4×10⁵ cells) were transferred to 96-well plates for incubation in triplicate with anti-CD4 (10 μg/mL), anti-CD62L (30 μg/mL), or isotype (30 μg/mL) antibody at 37° C. for 60 minutes prior to the addition of virus. Luciferase viruses pseudotyped with envelopes from R5- and X4-tropic HIV-1 and VSV were added to the cells at a concentration of 100 ng/mL HIV p24. The infected CD8− PBMCs were then incubated for 72 hours, lysed, and assayed for luciferase activity according to manufacturer's recommendations (Promega Corporation; Madison, Wis.). Pseudotyped virus from GNTI⁻ cells were added at 100 ng/mL to cells as above.

### HIV-1_{Bal} Infection

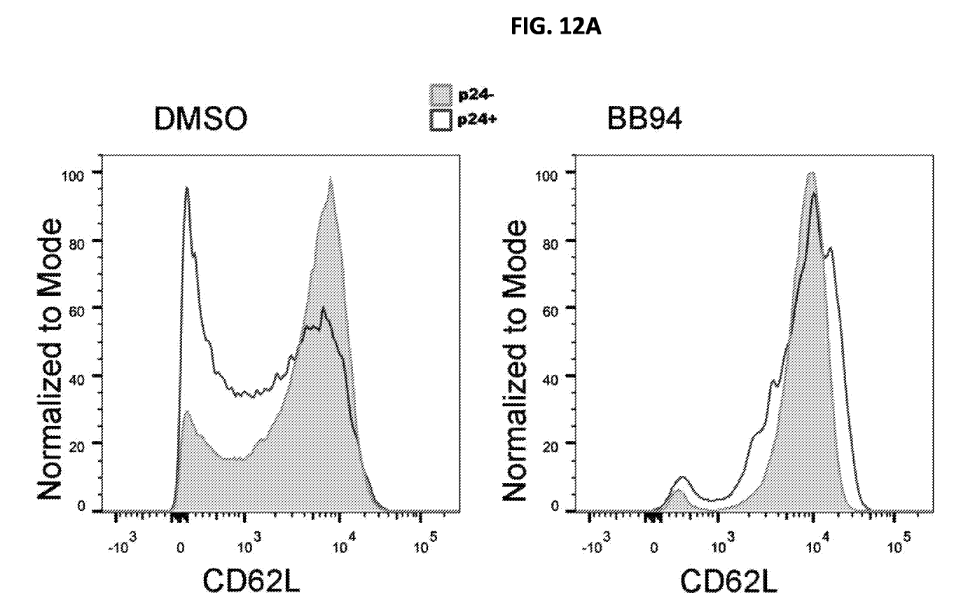

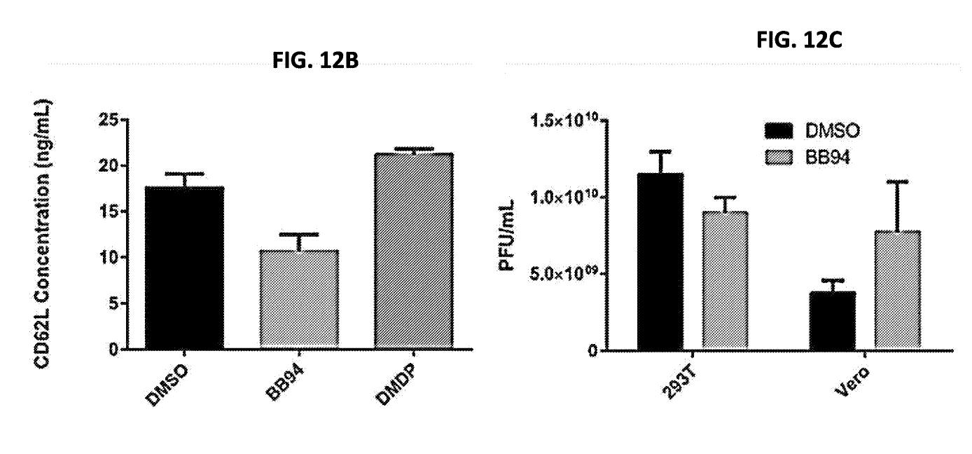

Activated CD8-depleted PBMCs were resuspended at 2×10⁶/mL in culture media. One mL cells were incubated with antibodies or inhibitors at 37° C. for 60 minutes prior to infection. The concentrations used were: 10 μg/mL antiCD4, 30 μg/mL anti-CD62L, 200 μg/mL T20, 10 μg/mL isotype antibody, 5 mM EDTA and 10 μM BB-94. BB-94 and EDTA treatment did not affect the viability of PBMC (FIG. 12). Cells were exposed to HIV-1_{BaL} at an optimal dilution of 1:5000 stock virus (˜80 TCID₅₀), unless otherwise specified, for 1 hour at 37° C. followed by washing with 10 mL culture media. Culture supernatants were collected and wells were replenished with fresh media on days 3 and 6 post-infection. Intracellular p24 levels were measured using phycoerythrin (FITC) conjugated KC57 antibody using the CYTOFIX/CYTOPERM™ kit from BD Biosciences (San Jose, Calif.). Samples were collected on a BD FACSCanto II. Real time PCR was assayed as described previously (Chun et al., J Infect Dis, 208, 1443-1447, 2013). All statistical analyses were carried out using the software Prism 6 (GraphPad Software, Inc.). The inhibitors were added 30 minutes before infection and were replenished after the post infection wash and subsequent media exchanges.

### PNGase Treatment of Activated CD8-Depleted PBMCs

HIV-1_{BaL} virus was diluted to 1:5000 in RPMI 1640 containing 0.5% FBS. The virus was incubated with 20,000 U PNGase/ml for one hour at 37° C. Mock PNGase-treated virus was exposed to the low FBS environment required for PNGase activity but without PNGase. Activated CD8-depleted PBMCs were resuspended at 2×10⁶/mL in the PNGase-treated, or control virus dilutions. Cells were infected for one hour at 37° C. as previously described. The CD8− PBMCs were then washed with R10 and resuspended at 2×10⁶ cells/ml and plated in a 48 well plate. Infection and analysis as previously described.

### Central Memory CD4 T Cell Staining

On Day 6 or Day 11 post infection, cells were harvested and stained with T cell memory surface markers including CD3, CD4, CD27, CD45RO, CD62L, CCR7, or the appropriate isotype controls. Cells were washed, permeabilized using the BD CYTOFIX/CYTOPERM™ kit (BD Biosciences) according to manufacturer's instructions and stained for intracellular p24. Samples were acquired on the BD FACSCANTO™ and analyzed using FLOWJO™ software.

### Stimulation for IFNγ Production

On Day 6 post infection, CD8-depleted PBMCs were stimulated for 6 hours with Leukocyte Activation Cocktail (BD Biosciences) at 37° C. Cells were stained for memory cell markers as above, permeabilized with the BD CYTOFIX/CYTOPERM™ kit, then washed and stained for intracellular p24 and IFN-γ. Samples were acquired on the BD FACSCANTO™ and analyzed using FLOWJO™ software.

### Cell-Cell and Cell-Free TZM-BL/62L Assay

Neomycin-resistant vectors from GeneCopoeia (Rockville, Md.) containing coding regions for human CD62L were transfected into TZM-BL cells using LIPOFECTAMINE™ 2000 from Invitrogen (Carlsbad, Calif.). Transfected cells were selected using 300 μg/mL G418 and passaging as needed for two weeks. Single colonies were isolated by limiting dilution in a 96-well plate and expanded before G418 was removed. Stable expression was analyzed and clone 1, with the highest level of expression, was used in all future analyses and is henceforth referred to as TZM-62L.

For the cell-cell transfer assay, TZM-BL and TZM-62L cells were seeded in a 96-well, flat-bottom plate at 3,000 cells/well three days before the assay. Three days post infection with HIV-1_{BaL}, activated CD8-depleted PBMCs with and without the presence of BB-94 were added to the wells at the concentration of 80×10³, 40×10³, 20×10³, 10×10³, and 5×10³ cells/well with the volume of each well equalized using the CD8− PBMC media. Additional BB-94 was added to both the cells infected in the presence of BB-94 and a sample of cells that only received BB-94 treatment for the cell-cell transfer assay. All conditions were prepared in triplicate. The cell mixtures were incubated at 37° C. for three days, followed by lysis and measurement of the subsequent luciferase expression as per the manufacturer's instructions.

For the cell-free infection assay, TZM-BL and TZM-62L cells were seeded as above. Three days post infection with HIV-1_{BaL}, the supernatant from infected activated CD8-depleted PBMCs were added to the 96-well plates containing TZM-BL or TZM-62L cells and titrated across the plate in two-fold dilutions for 10 dilutions. All conditions were prepared in triplicate. Following incubation as for the cell-cell transfer assay, the cells were lysed and the luciferase activity was measured per the manufacturer's instructions.

### HIV-1 Release Assay

PBMCs were obtained by leukapheresis and ficoll-hypaque centrifugation. CD4+ T cells were isolated using a cell separation system (StemCell Technologies). Cells were cultured with medium alone or with plate-bound anti-CD3 and soluble anti-CD28 antibody in the absence (DMSO) or presence of BB-94 in duplicate for 48 hours. The copy number of virion-associated HIV RNA in the above cell culture supernatants was determined using the COBAS™ Ampliprep/COBAS™ TAQMAN™ HIV-1 Test, Version 2.0 (Roche Diagnostics). The limit of detection for this system is 20 copies/ml.

### Confocal Microscopy

CD4+ T cells used for co-localization of CD62L and CD4 were prepared from isolated PBMCs using the StemCell EASYSEP™ Human CD4⁺ T Cell Enrichment Kit. Isolated CD4+ cells were then spun onto Superfrost glass slides using a CYTOSPIN™ 3 and CYTOSEP™ funnels (Fisher Scientific, Hampton, N.H.) at 1000 rpm for 3 minutes followed by fixation in 90% methanol and stained in 1×PBS with 10% FBS and 0.03% NaN₃. Alexa-647 labeled CD62L antibody and FITC, phycoerythrin (PE) or biotin labeled CD4 antibody was used in a 1:250 dilution for 15 minutes followed by two washes. For biotin labeled slides, streptavidin conjugated Alexa-405 was added to the staining mix. Labeled slides were mounted with PROLONG™ Gold Antifade Reagent (Life Technologies, Grand Island, N.Y.) and sealed after 24 hour curing with nail polish. 16-bit images were captured on a Zeiss LSM 780 AxioObserver confocal microscope using a 63×/1.40 oil immersion DIC M27 objective and Zeiss Zen 2012 Black Edition software. A 405 nm diode laser and 633 nm diode laser were used to excite Alexa-405-conjugated CD4 antibody and Alexa-647-conjugated CD62L antibody, respectively. The diffraction grating was set to capture peak emission for Alexa-405 and Alexa-647 (452 nm and 668 nm). Colocalization analysis was done in FIJI (Schindelin et al., Nat Methods 9, 676-682, 2012) using the co-localization plugins Coloc_2 and JACoP.

### Total Internal Reflection Fluorescence Gp120-Qdot Preparation

gp120-QDots were prepared by combining QDOT™ 625 ITK™ carboxyl quantum dots, 12-fold molar excess monomeric gp120, and excess EDC and NHS in 1×PBS with 10 mM HEPES. After 2 hours at room temperature and overnight at 4° C., the reaction was quenched with 1M Tris (final concentration 300 mM) and stored at 4° C. until used. The final concentration used in the assay was 27 μM. Full conjugation of gp120 to the Qdots was examined by SDS-PAGE using the Pierce Silver Stain Kit from Thermo Fischer Scientific (Rockford, Ill.). Anti-CD4 (RPA-T4), anti-CD62L (DREG-56), and isotype (IgG1) were used at 10, 30 and 10 μg/mL respectively as in all other experiments.

HeLa cells expressing selectins were grown in NUNC™ 6-well UPCELL™ plates from Thermo Scientific (Waltham, Mass.) at 37° C. and 5% CO₂ in DMEM/F12 supplemented with 5% FBS, 1% penicillin/streptomycin, and 300 μg/mL G418. Cells were gently released from the surface at room temperature using versene and slow pipetting. Suspended cells were transferred to ethanol cleaned 8-well glass coverslip chambers and allowed to adhere for 16 to 48 hours before used. Qdots were added to the HeLa cells and allowed to equilibrate before imaging. Images were collected on an Olympus IX-81 microscope adapted for TIRF imaging. A 405 nm diode laser was used to excite Qdots and emission was filtered with a 605/40 band-pass filter before imaging on a Cascade IIB 1024EM CCD camera. To obtain a larger fluorescent depth of field, the TIRF laser was adjusted to enter the sample and travel parallel to the surface of the glass coverslip for an acute angled illumination. Imaging of the labeled cells was collected using METAMORPH™ (Molecular Devices LLC, Sunnydale, Calif.) by live-streaming a series of 100 nm-step focal depths of both DIC as well as fluorescent images. Image stacks were deconvoluted with a measured PSF in Huygens Essential software by Scientific Volume Imaging (Hilversum, Netherlands), followed by Qdot recognition and quantification using FIJI imaging software (Schindelin et al., Nat Methods 9, 676-682, 2012) and its 3D object counter. For immobilized protein binding, 100 ng of soluble CD4 or CD62L were adsorbed overnight at room temperature to 8-well sterile glass coverslip chambers. The wells were washed twice before the addition of QDots in 1×PBS containing 10% FBS. After room temperature equilibration for at least 10 minutes, the QDots were imaged as above.

### Example 2: HIV Targets CD62L on Central Memory T Cells Through Viral Envelope Glycans for Adhesion and Induces Selectin Shedding for Viral Release

This example describes the finding that shedding of CD62L on T cells is required for efficient release of HIV from infected cells.

HIV-1 gp120 Recognizes CD62L in Solution

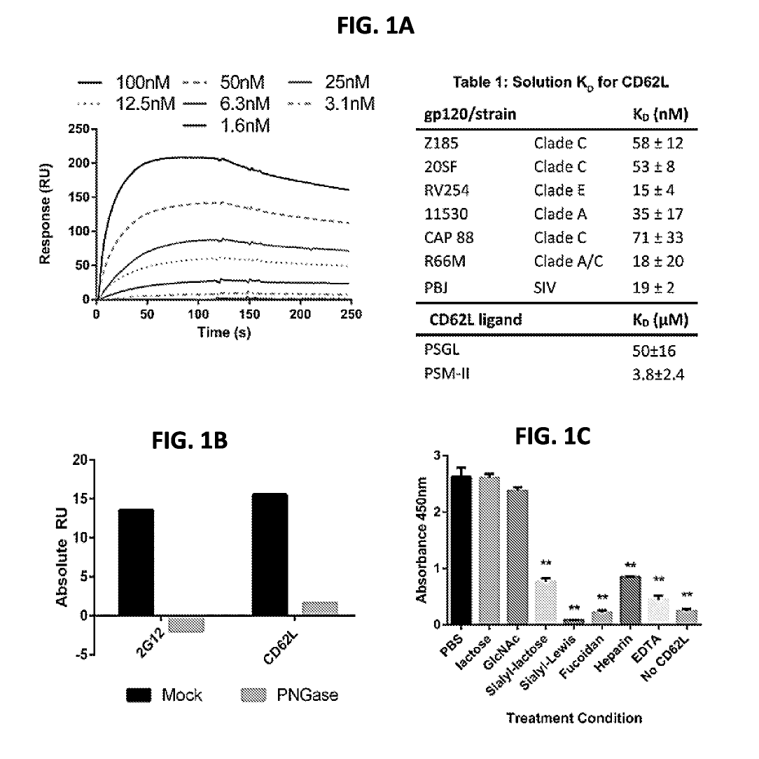

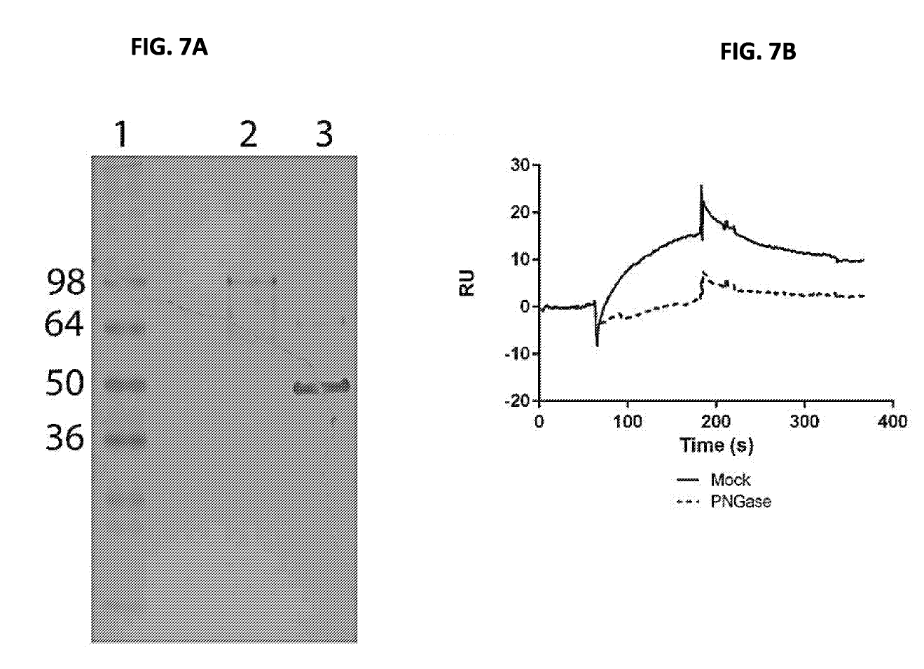

HIV-1 envelope gp120 is highly decorated with N-linked glycans (Doores et al., Proc Natl Acad Sci USA 107, 13800-13805, 2010). The known ligands for CD62L are sialyl-Lewis X (sLe^{x})-like O-linked glycan present on PSGL-1 and mucins (Klopocki et al., J Biol Chem 283, 11493-11500, 2008). Although such carbohydrates are found on N-linked glycans (Mitoma et al., Nat Immunol 8, 409-418, 2007), they have not been observed on HIV-1 gp120. To determine if CD62L recognizes carbohydrates on gp120, surface plasmon resonance (SPR) binding experiments were carried out between soluble human L-selectin (sCD62L) and a recombinant gp120 from strain 20SF of Clade C. The gp120 bound to immobilized CD62L with 53 nM affinity (FIG. 1A). Additional binding studies using gp120 from multiple strains of HIV-1 and SIV showed that all exhibited high affinity binding to CD62L, ranging from 10-60 nM, irrespective of R5 or X4 tropism (Table 1). In contrast, CD62L bound to soluble mucin and recombinant PSGL-1 with much lower affinities, 3.8 and 50 μM respectively, similar to what has been previously reported (Poppe et al., J Am Chem Soc 119, 1727-1736, 1997). The selectin and gp120 binding was dependent on the envelope N-linked glycans as peptide N-glycosidase F (PNGase F)-treated gp120 lost its binding to CD62L (FIG. 1B, FIG. 7).

TABLE 1
Solution K_{D} for CD62L

| gp120/strain | Clade | K_{D} (nM) |
|---|---|---|
| Z185 | Clade C | 58 ± 12 |
| 20SF | Clade C | 53 ± 8 |
| RV254 | Clade E | 15 ± 4 |
| 11530 | Clade A | 35 ± 17 |
| CAP 88 | Clade C | 71 ± 33 |
| R66M | Clade A/C | 18 ± 20 |
| PBJ | SIV | 19 ± 2 |

| CD62L ligand | K_{D} (μM) |
|---|---|
| PSGL | 50 ± 16 |
| PSM-II | 3.8 ± 2.4 |

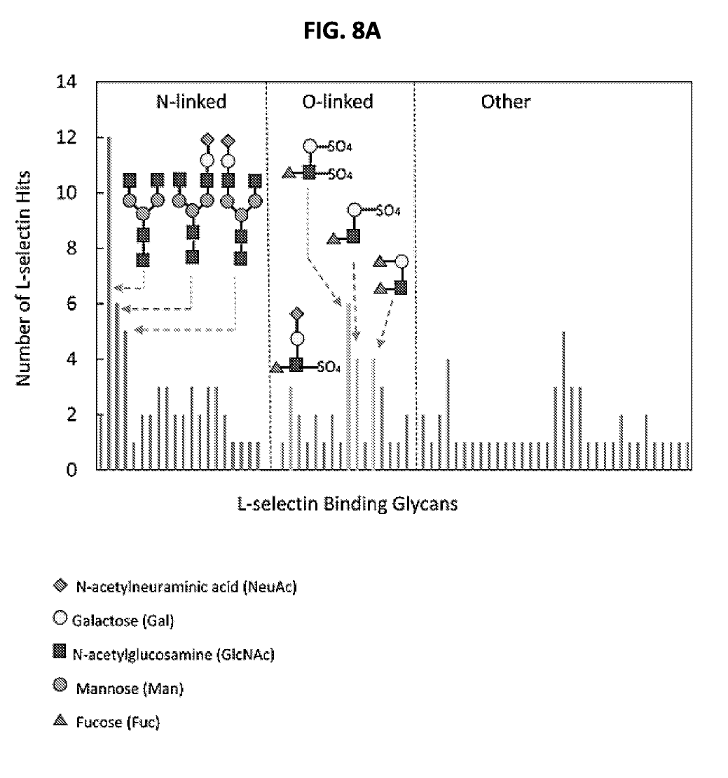

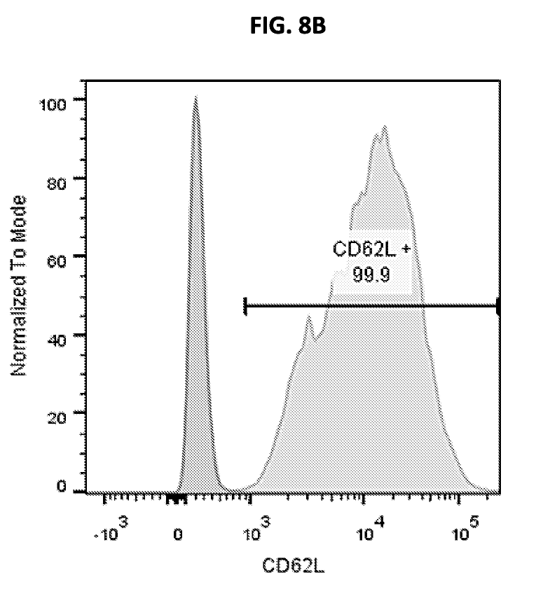

To further characterize the specificity of this CD62L recognition, binding to gp120 was carried out in an ELISA assay in the presence of EDTA and various carbohydrates (FIG. 1C). The results showed that the gp120-CD62L binding was calcium dependent, consistent with the C-type lectin properties of CD62L. Heparin, fucoidan and sialyl-Lewis X, known ligands of CD62L, competed with gp120 for receptor binding, suggesting that CD62L recognizes gp120 in a manner similar to other selectin ligands. Furthermore, gp120 binding was inhibited by sialyllactose, an analog of the terminal carbohydrates on complex N-linked glycans, but not by lactose or N-acetylglucosamine. To further address the potential of L-selectin binding of N-linked glycans, all L-selectin glycan array data from the Consortium for Functional Glycomics database was analyzed. The combined glycan array profiles clearly showed human L-selectin recognition of carbohydrates from hybrid and complex N-linked glycans in addition to their known sulfo-sialyl-Lewis-X type of O-linked glycans (FIG. 8).

HIV-1 gp120 Recognizes CD62L on CD4⁺ T Cells

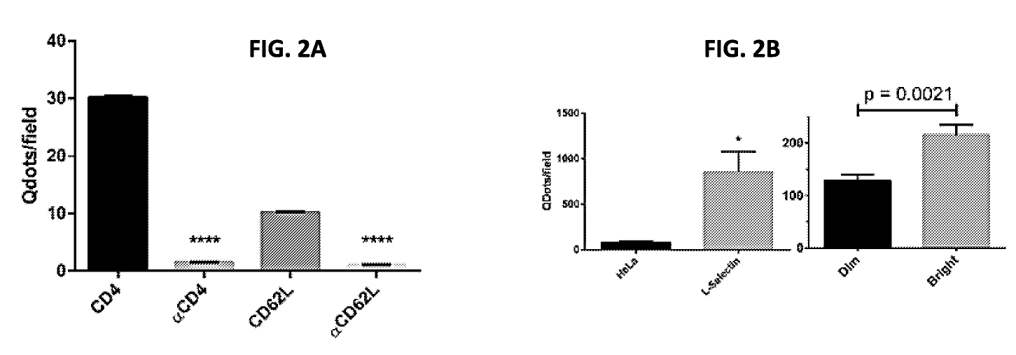

To investigate if gp120 recognized cell surface expressed CD62L, recombinant HIV-1 gp120 was conjugated to fluorescence Qdots at approximately 10 gp120 per Qdot, mimicking the number of envelope trimers on HIV-1 virus (Zhu et al., Nature 441, 847-852, 2006). Fluorescent gp120-Qdots bound to immobilized recombinant CD62L or CD4, as shown by TIRF microscopy (FIG. 2A). Further, these gp120-Qdots bound to CD62L-transfected HeLa cells with their binding level correlated with that of CD62L expression (FIG. 2B). On CD4⁺ T cells, CD62L and CD4 exhibited similar surface distributions, suggesting a potential co-engagement of the two receptors by an HIV-1 virion (FIG. 2C). To better emulate an in vivo interaction, CD4⁺ T cells were incubated with gp120-Qdots and binding was analyzed using flow cytometry. The observed binding was reduced in the presence of either anti-CD62L or anti-CD4 antibodies and was further reduced when both CD62L and CD4 antibodies were present (FIG. 2D). These findings suggest that CD62L- and CD4-mediated binding were independent and additive.

### CD62L Facilitates Pseudo-HIV and HIV-1_{BaL} Virus Infections

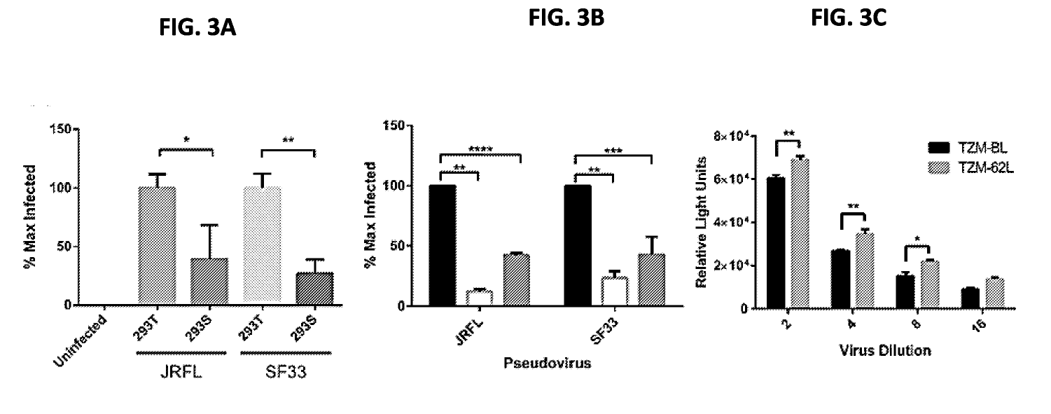

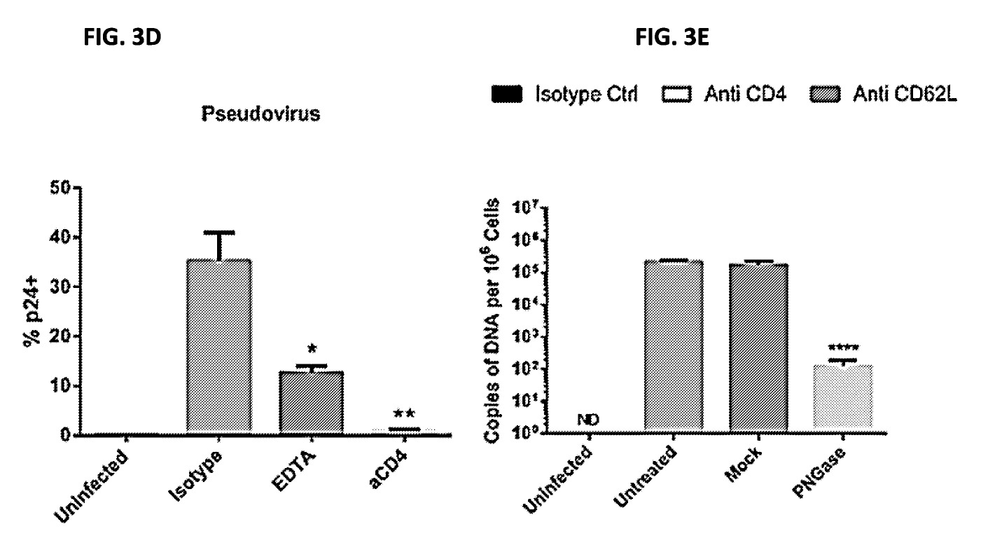

To investigate whether gp120 recognition by CD62L modulated HIV-1 infections in vitro, pseudotyped R5 (JRFL) or X4 (SF33) tropic HIV-1 luciferase viruses were produced in HEK 293T cells or HEK 293S GnTI⁻ cells, which are deficient for N-acetyl-glucosaminyltransferase I and thus lack mature complex N-glycans (Reeves et al., Proc Natl Acad Sci USA 99, 13419-13424, 2002). Activated CD8-depleted PBMCs infected with equal amounts of pseudotyped HIV-1 show that glycan deficient viruses from HEK 293S GnTI⁻ cells infected less than their glycan sufficient counterparts (FIG. 3A). This illustrates the preference of complex viral glycans in HIV-1 infection and is consistent with recent findings that cells from glycosylation deficient individuals were resistant to HIV-1 infection (Sadat et al., N Engl J Med 370, 1615-1625, 2014). Furthermore, the infections of CD4⁺ T cells with both JRFL- and SF33-pseudotyped HIV-1 were significantly reduced in the presence of a lectin-binding blocking CD62L antibody (DREG-56) when compared to an isotype control (FIG. 3B), demonstrating a direct role of CD62L in HIV-1 infection of CD4⁺ T cells. The marked reduction of infection by blocking CD62L, especially with CD4 accessible, suggests that CD62L functions as a viral adhesion receptor facilitating HIV-1 recognition of CD4.

To evaluate the role of this lectin-gp120 binding in replication competent HIV-1 infection, CD62L was transfected into an HIV-1 reporter cell-line, TZM-BL, and a stable CD62L-expressing transfectant referred to as TZM-62L was established (FIG. 8). HIV-1_{BaL} was able to infect TZM-62L cells consistently better than the untransfected parental cells (FIG. 3C), supporting the role of CD62L in facilitating HIV-1 adhesion and infection. HIV-1_{BaL} infection of activated CD8-depleted PBMCs in the presence of 5 mM EDTA, which eliminates the calcium dependent binding of CD62L to gp120 (FIG. 1C), was then investigated. EDTA significantly reduced HIV-1_{BaL} infection of the cells, as measured by the content of intracellular viral capsid p24 (FIG. 3D). Further, removing HIV-1_{BaL} envelope N-linked glycans with PNGase F significantly reduced infection by HIV-1_{BaL} compared to the mock-treatment, as measured by a decrease in copies of HIV-1 DNA per 10⁶ cells (FIG. 3E), demonstrating the involvement of viral glycans in HIV-1_{BaL} infection of primary CD4 T cells.

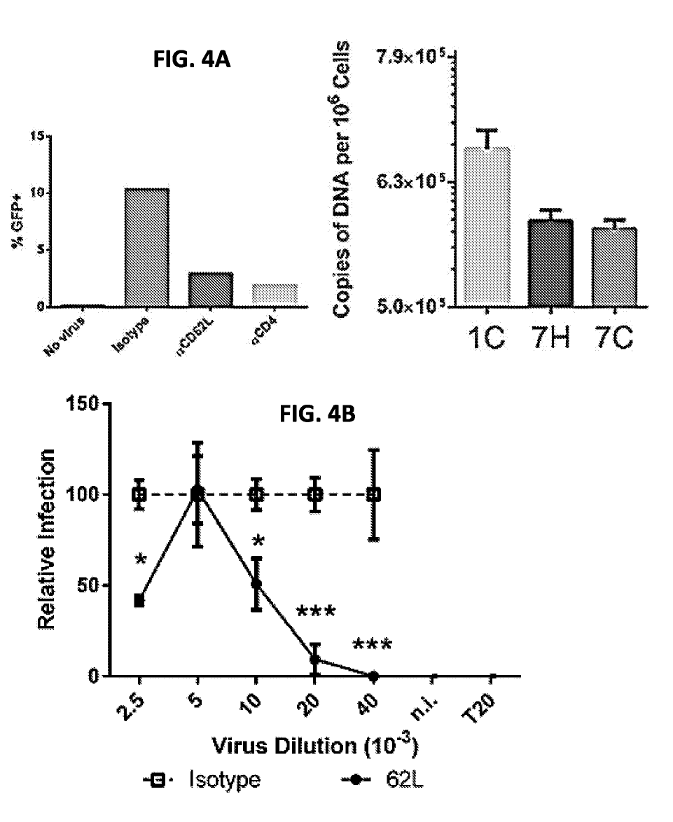

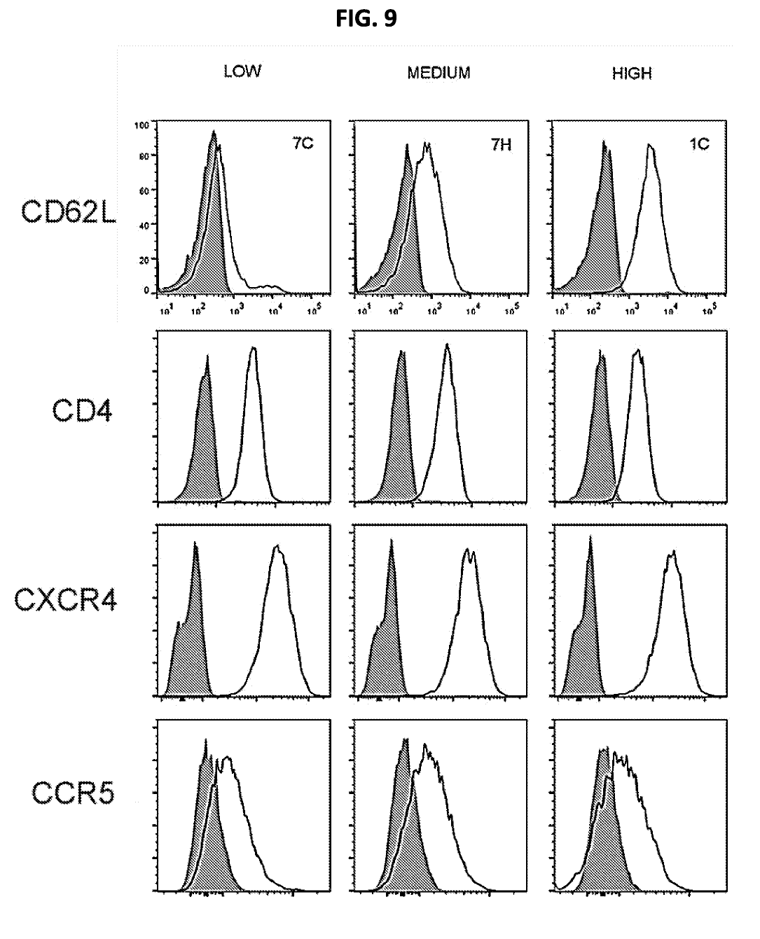

To examine the role of CD62L in replication competent HIV-1 infection of CD4⁺ T cells, HIV-1_{BaL} infections of CD4⁺, CD62L⁺ Rev-CEM cells that use GFP as a reporter for HIV-1 infection (Wu et al., Curr HIV Res 5, 394-402, 2007) were carried out. The infection of Rev-CEM cells by HIV-1_{BaL} was significantly inhibited by either anti-CD4 or anti-CD62L blocking antibodies (FIG. 4A, left panel). Rev-CEM cells were then subcloned by limiting dilution and stable clones, 1C, 7H and 7C, with high, medium and low CD62L expression respectively, were generated (FIG. 9). All clones expressed similar levels of CD4, CXCR4 and CCR5 (FIG. 9). Rev-CEM cells expressing a high level of CD62L showed a significantly higher level of HIV-1_{BaL} infection when compared to clones expressing lower levels of CD62L (FIG. 4A, right panel). To further evaluate the contribution of CD62L to HIV-1_{BaL} infection, activated CD8-depleted PBMCs were infected with decreasing amounts of virus in the presence of anti-CD62L blocking antibody. The presence of CD62L antibody significantly inhibited HIV-1_{BaL} infection, particularly at lower virus concentrations (FIG. 4B). Similar blocking effects of anti-CD62L antibody were observed with infections of titrated JRFL and SF33 pseudovirus in activated CD8-depleted PBMCs (FIGS. 4C and 4D). Collectively, these results demonstrate the involvement of CD62L in viral entry.

### HIV-1 Infection Resulted in CD62L Shedding on Infected Cells

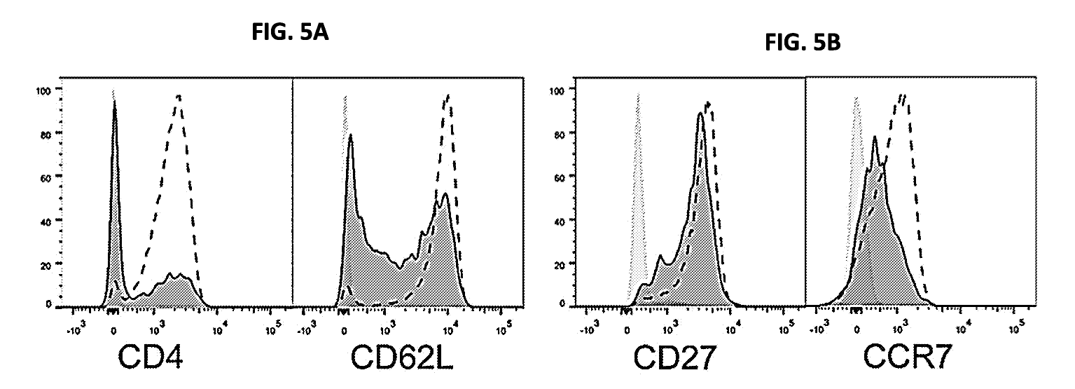

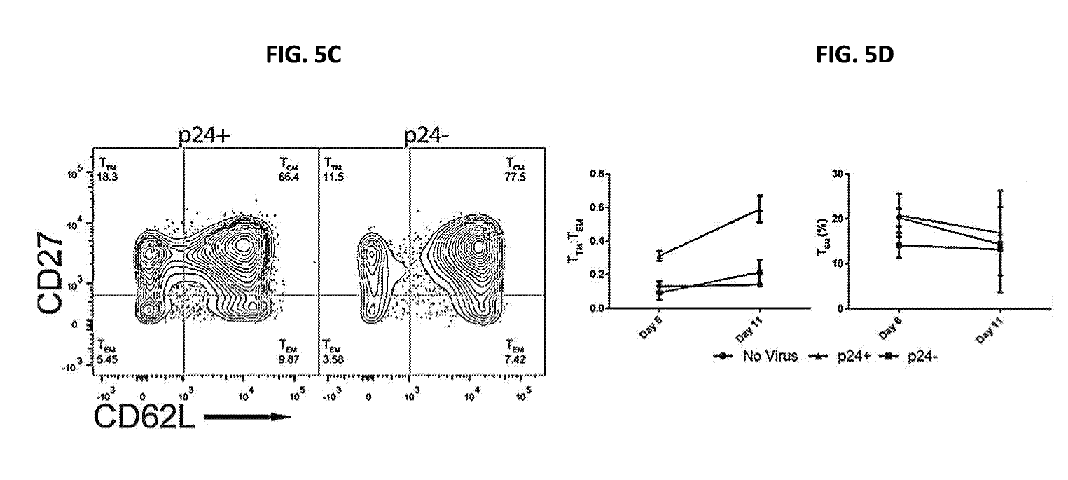

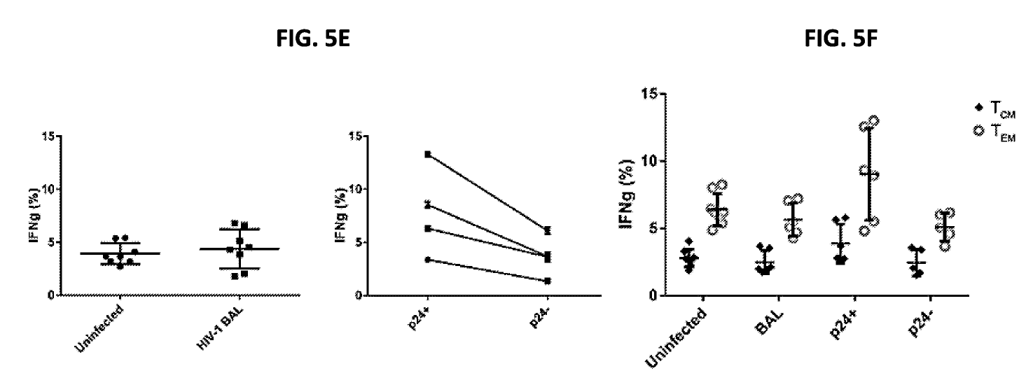

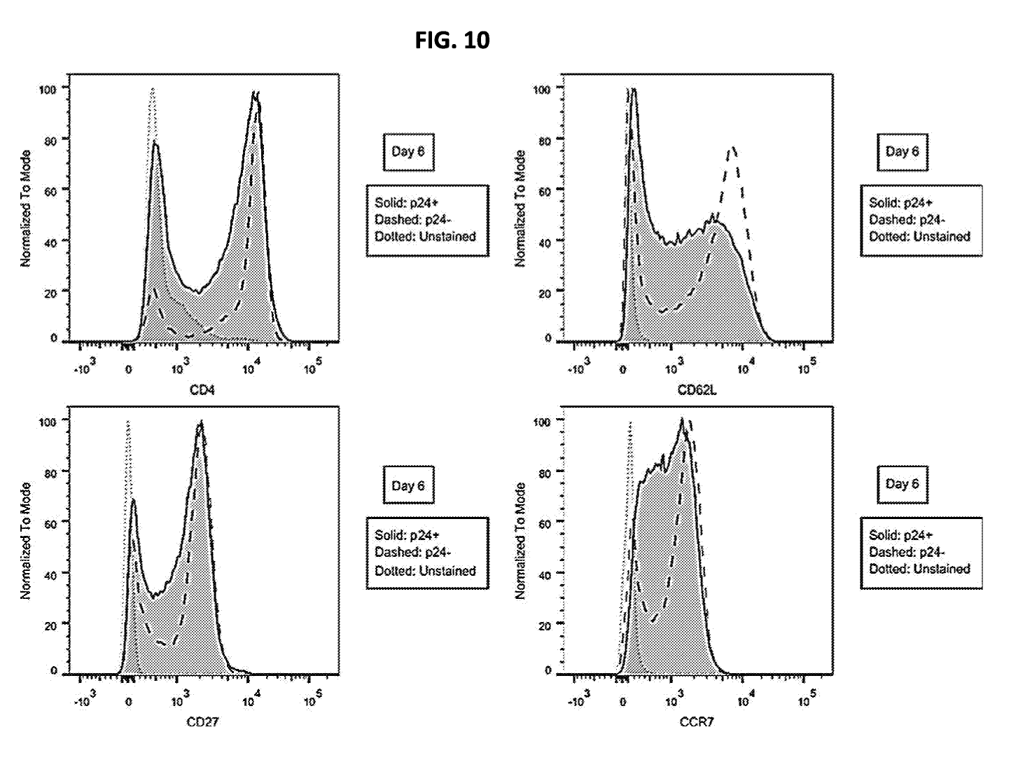

CD62L is known to shed from activated leukocytes and T cells, which is associated with the differentiation of central memory to effector memory T cells and allows them to exit lymph nodes to migrate to peripheral sites of inflammation (Galkina et al., J Exp Med 198, 1323-1335, 2003). In addition, crosslinking of CD4 with HIV-1 envelope induced CD62L shedding on resting CD4⁺ T cells (Wang et al., Blood 103, 1218-1221, 2004). Accordingly, it was investigated if HIV-1_{BaL} infection induces CD62L shedding on infected CD4⁺ T cells. Activated CD8-depleted PBMCs were infected with HIV-1_{BaL} for a total of 11 days. On days 6 and 11, intracellular p24 staining was performed along with surface staining for CD62L, CD3, and CD4. At day 11, approximately 30% of the PBMCs were positive for p24. Consistent with published data (Garcia et al., Nature 350, 508-511, 1991; Guy et al., Nature 330, 266-269, 1987; Vassena et al., J Virol 89, 5687-5700, 2015; Trinite et al., PLoS One 9, e110719, 2014), CD4 expression was decreased at both day 6 and day 11 (FIG. 10 and FIG. 5A, respectively). Similarly, infected p24⁺ T cells showed reduced CD62L expression compared to the p24⁻ population (FIG. 10, FIG. 5A). While a significant number of p24⁺ T cells lost both CD4 and CD62L expressions, similar percentages of the infected T cells were either CD4⁺ CD62⁻ or CD4⁻ CD62⁺, suggesting that downregulation of CD62L occurs independent of CD4 internalization.

### CD62L Shedding Leads to the Loss of the Central Memory Subpopulation, but Infected T Cells Remain Competent in Cytokine Production

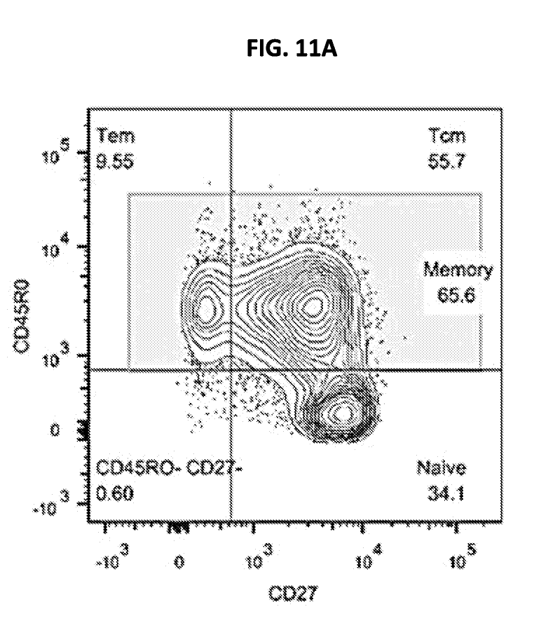

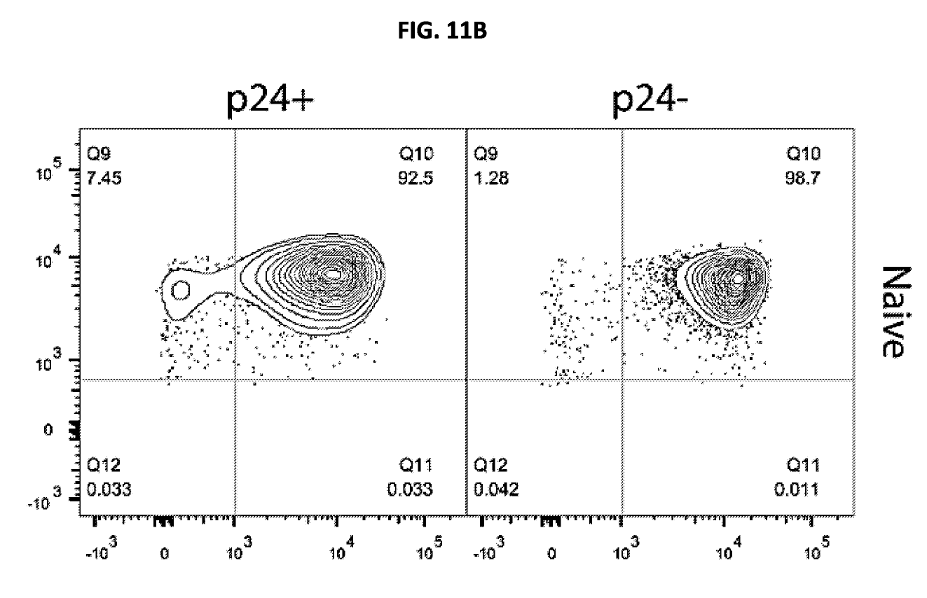

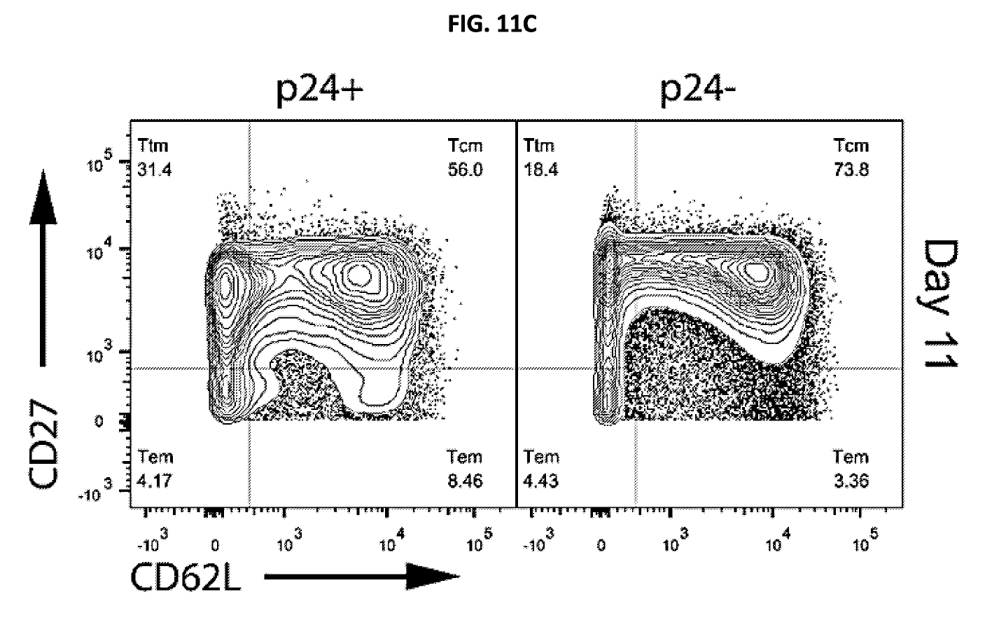

HIV-1 preferentially infects memory CD4⁺ T cells, especially central memory CD4⁺ T cells (Brenchley et al., J Virol 78, 1160-1168, 2004; Schnittman et al., Proc Natl Acad Sci USA 87, 6058-6062, 19901 Holl et al., Arch Virol 152, 507-518, 2007; Chomont et al., Nat Med 15, 893-900, 2009; Lambotte et al., Aids 16, 2151-2157, 2002). The ability to maintain or recover the central memory T cell population is a hallmark of HIV-1 control and successful response to antiviral treatment (Potter et al., J Virol 81, 13904-13915, 2007; Munoz-Calleja et al., Aids 15, 1887-1890, 2001). Naïve (CD45R0⁻, CD62L⁺) CD4⁺ T cells have also been shown to be susceptible to HIV-1 infection (Ostrowski et al., J Virol 73, 6430-6435, 1999). To investigate if HIV-1 infection-induced CD62L shedding contributes to the loss of central memory CD4⁺ T cells in patients, HIV-1_{BaL} infected, activated CD8-depleted PBMCs with memory markers were labelled using CD3, CD27, CCR7, CD62L, CD4 and CD45RO antibodies (FIG. 11). On day 6 post-infection, a majority of infected memory (CD45RO⁺) T cells were CD27⁺, CD62L⁺ central memory T cells (T_{CM}) (FIG. 11). Likewise, a majority of the infected naïve population expressed CD62L as well. HIV-1_{BaL} infection, however, resulted in a significant increase in the number of CD45RO⁺, CD27⁺, CD62L⁻ transitional memory T cells (T_{TM}) in the infected p24⁺ versus p24⁻ population (FIG. 5C). The p24⁺ naïve population also showed an increase in CD62L⁻, CD27⁺ population when compared to the p24⁻ population (FIG. 11B). As the infection proceeds to day 11, T_{TM} is further increased with a concomitant decrease in the number of T_{CM} (FIG. 11C). The infection-induced memory cell transitioning from T_{CM} to T_{TM} is evident from a significant higher ratio between the number of T_{TM} and T_{CM} cells associated with the p24⁺ versus p24⁻ and uninfected memory T cells (FIG. 5D). The ratio of T_{TM} T_{CM} is further increased from day 6 to 11 (FIG. 5D). In contrast, the number of CD45RO⁺, CD27⁻ effector memory (T_{EM}) cells remain similar in the p24⁺ compared to the p24⁻ population on both days 6 and 11 (FIG. 5D). Thus, HIV-induced CD62L shedding resulted in a preferential loss of the T_{CM} population, which is consistent with clinical observations.

Previously it has been shown that the effector memory CD4⁺ T cells from HIV-1 patients are dysfunctional as they appear to be immaturely differentiated and produce a low amount of cytokines (Yue et al., J Immunol 172, 2476-2486, 2004; French et al., HIV medicine 8, 148-155, 2007; Younes et al., J Exp Med 198, 1909-1922, 2003). To investigate if this effector T cell dysfunction occurs in infected T cells, and if it is related to CD62L shedding, activated CD8− depleted PBMCs infected with HIV-1_{BaL} were stimulated for IFN-γ production. Overall, the infected T cells produced similar amounts of IFN-γ when compared to uninfected cells (FIG. 5E). When comparing the p24⁺ and p24⁻ T cells within each infection, the infected p24⁺ cells consistently produced more cytokine (FIG. 5E). These data suggest that the infected T cells are competent in cytokine production. When cytokine production was measured for each subset of memory T cells, T_{CM} produced less IFN-γ than T_{EM} in uninfected controls (FIG. 5F), consistent with previous publications (Sallusto et al., Nature 401, 708-712, 1999).

Since HIV-induced CD62L shedding results in a preferential loss of central memory CD4⁺ T cells, it was investigated whether the apparent dysfunctional effector memory phenotype is associated with the infection-induced loss of central memory markers. In addition to the loss of CD62L expression, a partial downregulation of CCR7, and to a lesser extend CD27, was also evident in p24⁺ cells on day 11 post infection when compared to the p24⁻ population (FIG. 5B). HIV-1 infection therefore resulted in the partial downregulation of at least two of the three central memory T cell markers. The consequence of this downregulation is that these central memory cells have an effector memory appearance based on CD62L or CCR7 expressions. As a result, the observed IFN-γ from these “effector memory T cells” would be lower compared to uninfected effector memory T cells, since central memory T cells produce less cytokine than effector memory T cells. However, if memory T cells are delineated based on the most stable marker CD27, similar amounts of IFN-γ were observed between the infected and uninfected T cells for both central and effector memory cell types (FIG. 5F). This data suggests that HIV-1 infected CD4⁺ T cells are not defective in cytokine production.

### CD62L Shedding is Required for HIV Viral Release

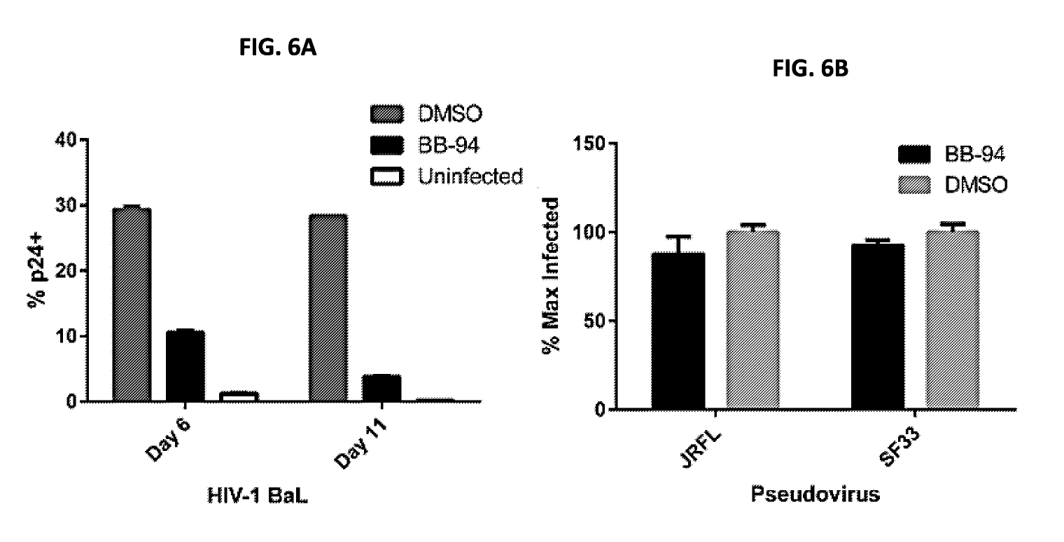

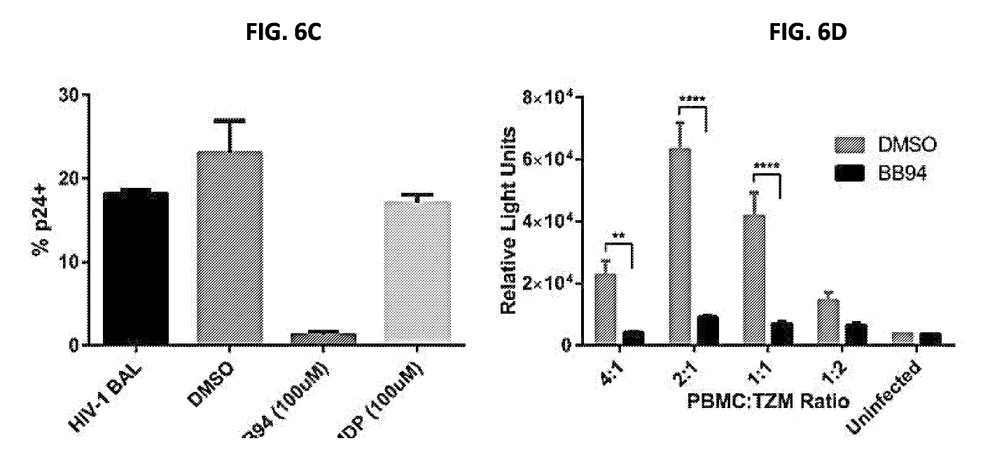

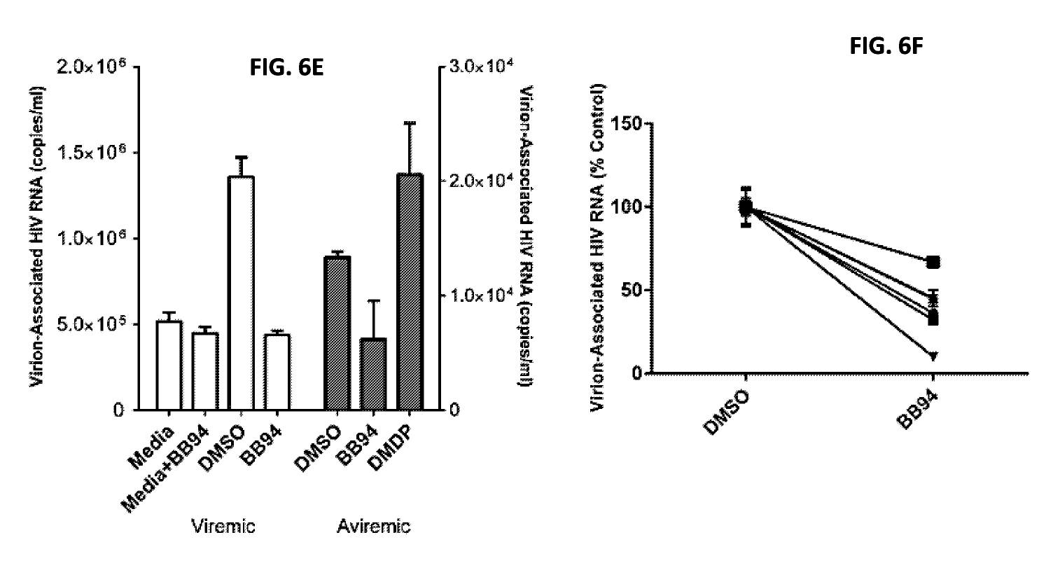

To investigate if HIV-induced CD62L shedding on infected T cells affects viral pathogenesis, studies were conducted to inhibit CD62L shedding with a metalloproteinase inhibitor. While the protease responsible for HIV-induced CD62L shedding remains to be defined, the shedding of CD62L on activated T cells is primarily mediated by ADAM17 (Le Gall et al., Mol Biol Cell 20, 1785-1794, 2009), and can be inhibited by Batimastat (BB-94) (Koolwijk et al., Blood 97, 3123-3131, 2001; Wang and Sun, PLoS One 9, e91133, 2014; Peschon et al., Science 282, 1281-1284, 1998). In these studies, BB-94 significantly inhibited CD62L shedding from infected (p24⁺) compared to their p24⁻ population of CD4⁺ T cells (FIG. 12). It was expected that the inhibition of CD62L shedding from activated T cells would facilitate L-selectin mediated viral adhesion and entry. Instead, HIV-1_{BaL} infection of CD4⁺ T cells was reduced by 60% at day 6 and 80% at day 11 in the presence of 5 μM BB-94 (FIG. 6A). The marked inhibition of HIV-1_{BaL} infection by BB-94 indicates a potential role of CD62L shedding in viral release as the shedding occurs preferentially in infected T cells (FIG. 5).

To separate the possible effect of BB-94 in HIV-1 entry from its role in release, single-round-infection of JRFL and SF33 pseudovirus was used to measure its effect on entry alone. In contrast to HIV-1_{BaL} infection, BB-94 did not have a significant effect on the JRFL and SF33 pseudovirus infections (FIG. 6B), which supports the role of BB-94 in HIV-1 release. To address if the effect of BB-94 on HIV-1 infection is due to its adverse inhibition of other metalloproteinases important for T cell or viral function, instead of CD62L shedding, the effect of a related metalloproteinase inhibitor, dichloromethylenediphosphonic acid (DMDP) was tested for both CD62L shedding and HIV-1 infection. BB-94 and DMDP share overlapping specificity and both are inhibitors of matrix metalloproteinase (MMP)-1. However, BB-94 significantly inhibited CD62L shedding and HIV-1_{BaL} infection whereas DMDP showed no effect at 100 μM concentration (FIG. 12, FIG. 6C). The inhibitory effect of BB-94 also appeared to be dependent on the virus, as it did not affect VSV infection of 293T or Vero cells (FIG. 12).

Since there are largely two methods of viral infection, cell-free and cell-cell, the effect of BB-94 on HIV infection via cell-cell transfer was evaluated as a distinct and productive mode of infection (Pearce-Pratt et al., J Virol 68, 2898-2905, 1994; Jolly et al., J Exp Med 199, 283-293, 2004; Abela et al., PLoS Pathog 8, e1002634, 2012). When TZM-BL cells were incubated with activated CD8-depleted PBMCs infected with HIV-1_{BaL}, the presence of BB-94 resulted in an 80% reduction in the viral infection of TZM-BL cells compared to the control (FIG. 6D). Together these data show that CD62L shedding is required for viral release in both cell-free and cell-cell transfer HIV-1 infections. The profound inhibition of BB-94 to HIV infection revealed a new strategy for developing antiviral treatment. Currently, anti-HIV therapy consists of a combination of inhibitors specific for the viral protease, reverse transcriptase and entry. HIV viral release is a critical step in the virus life cycle and has not been previously targeted for therapy. The results disclosed herein indicate that targeting the viral release would be a new effective avenue against HIV infection in addition to the current regiment.

The investigation regarding the role of CD62L in viral release was expanded by using CD4⁺ T cells from both HIV-1 viremic and aviremic individuals. Here, CD4⁺ T cells were stimulated to release HIV-1 virus with an anti-CD3 antibody in the presence of BB-94. The extent of viral release was quantified as virion-associated HIV-1 RNA in the supernatant using an automated system (COBAS™ Ampliprep/COBAS™ TAQMAN™ HIV-1 Test Version 2.0, Roche Diagnostics). The viremic individuals were not receiving ART and had viral loads between 1,200 and 100,000 copies of HIV-1 RNA/mL, while individuals receiving ART had undetectable plasma viremia (<40 copies HIV RNA/mL). Anti-CD3 stimulated CD4⁺ T cells from viremic individuals produced 10-100 fold more virion-associated viral RNA than those from aviremic individuals (FIG. 6E). The presence of BB-94 profoundly inhibited the activation-induced viral release from both viremic and aviremic individuals, suggesting that shedding of CD62L is required for efficient HIV-1 release. The inhibition by BB-94 is specific as neither DMSO nor DMDP affected the viral release (FIG. 6E). The inhibition of CD62L shedding to HIV-1 viral release varies, however, between 30%-90% among infected individuals (FIG. 6F). These results indicate that HIV-1 induces CD62L shedding on infected T cells to promote the efficient release of progeny virus. One possible mechanism is that the envelope of a budding HIV-1 virion is retained through binding to CD62L, which is enhanced in the presence of BB-94 but not when the selectin is allowed to shed. Since the viral release from cells of HIV-1-infected aviremic individuals is likely from their persistent viral reservoir, the inhibition by BB-94 also suggests that CD62L is associated with a residual HIV-1 viral reservoir.

### Therapeutic Applications

L-selectin (CD62L) provides rolling adhesion for lymphocyte extravasation to secondary lymph nodes and sites of inflammation. The binding of the HIV-1 glycans to CD62L can be viewed similarly as viral rolling adhesion on CD4⁺ T cells. Adhesion of viral particles prior to CD4 binding is advantageous for viruses as this mechanism would allow it to sample the surface of T cell, which would enhance or facilitate infection. Biochemically, the recognition of gp120 by CD62L represents a novel function of the selectins as they are known to preferentially bind O-linked glycans. While CD62L prefers sialyl-Lewis X as individual carbohydrate ligands, the observed high affinity binding between CD62L and gp120, both in solution and on a cell surface, suggests the avidity of the binding from the highly glycosylated gp120 is important for CD62L recognition.

The effect of blocking CD62L was significant but generally less than that of blocking CD4, which is consistent with CD62L functioning as a viral adhesion receptor rather than an entry receptor. This is also consistent with the increased dependence on CD62L for HIV-1_{BaL} infection at lower viral concentrations.

The success of ART in suppressing plasma HIV-1 viremia has brought renewed focus on finding and eliminating latently infected viral reservoirs, which poses a major obstacle to the goal of achieving a cure (Eisele and Siliciano, Immunity 37, 377-388, 2012). While many cell types are productively infected by HIV-1 and could serve as a potential viral reservoir, results from patient studies suggest that resting memory CD4⁺ T cells constitute a major HIV-1 reservoir (Brenchley et al., J Virol 78, 1160-1168, 2004; Chomont et al., Nat Med 15, 893-900, 2009; Chun et al., Proc Natl Acad Sci USA 94, 1997; Chun et al., Nature 387, 183-188, 1997; Finzi et al., Science 278, 1295-1300, 1997; Wong et al., Science 278, 1291-1295, 1997). The inhibition of viral release from CD4⁺ T cells of both viremic and aviremic HIV-1-infected individuals by BB-94 illustrated for the first time that CD62L-expressing memory CD4⁺ T cells may constitute a major viral reservoir and that release of the virus requires proteolytic shedding of CD62L. Currently, there is no effective treatment to eliminate persistent HIV-1 reservoirs. One ongoing approach involves reactivation of latent HIV to purge the viral reservoirs (Archin et al., Nature 487, 482-485, 2012; Sgarbanti and Battistini, Curr Opin Virol 3, 394-401, 2013). At this time there are no antiviral drugs targeted at HIV-1 release, however BB-94 represents a new family of anti-HIV drugs for targeting viral release through the inhibition of metalloproteinases. If CD62L expression also marks a reservoir favored by HIV-1, similar to its preference in productive infection for CD62L-expressing central memory CD4⁺ T cells, inhibiting shedding of CD62L on resting memory CD4⁺ T cells could be used to eliminate viral release from this reservoir.

In summary, it is shown herein that HIV-1 interacts with CD62L on memory CD4⁺ T cells through its envelope glycans and uses this interaction for viral adhesion. Upon productive entry, the virus induces CD62L shedding on infected CD4⁺ T cells. The downregulation of CD62L, as well as other memory markers, results in a loss of infected central memory CD4 T cells and the mis-identification of infected central memory T cells as dysfunctional effector memory T cells. CD62L shedding is required for HIV release from the infected cells as the inhibition of the shedding reduced productive viral infection. This also holds true for activated CD4⁺ T cells derived from both viremic and aviremic individuals. These results implicate viral release as a new avenue for antiviral treatment with inhibitors targeted at CD62L shedding. Inhibitors such as BB-94 may constitute a new class of antiviral drugs for HIV-1 and other glycosylated viruses.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of inhibiting human immunodeficiency virus (HIV) release from an infected cell, comprising contacting the cell with an inhibitor of CD62L shedding, thereby inhibiting HIV release.

2. The method of claim 1, wherein the inhibitor is a metalloproteinase inhibitor.

3. The method of claim 2, wherein the metalloproteinase inhibitor is a matrix metalloproteinase (MMP) inhibitor.

4. The method of claim 2, wherein the metalloproteinase inhibitor is an ADAM family protein inhibitor.

5. The method of claim 4, wherein the inhibitor is an ADAM17 inhibitor, an ADAM10 inhibitor, or both.

6. The method of claim 1, wherein the inhibitor is a small molecule or an antibody.

7. The method of claim 6, wherein the small molecule inhibitor is batimastat.

8. (canceled)

9. The method of claim 1, which is an in vitro or ex vivo method.

10. The method of claim 9, wherein the cell is a T lymphocyte.

11. The method of claim 1, which is an in vivo method, wherein contacting the cell with an inhibitor of CD62L shedding comprises administering the inhibitor to a subject infected with HIV.

12. A method of treating a subject infected with HIV, comprising administering to the subject an inhibitor of CD62L shedding.

13. The method of claim 12, wherein the inhibitor is a metalloproteinase inhibitor.

14. The method of claim 13, wherein the metalloproteinase inhibitor is a matrix metalloproteinase (MMP) inhibitor.

15. The method of claim 13, wherein the metalloproteinase inhibitor is an ADAM family protein inhibitor.

16. The method of claim 15, wherein the inhibitor is an ADAM17 inhibitor, an ADAM10 inhibitor, or both.

17. The method of claim 12, wherein the inhibitor is a small molecule or an antibody.

18. The method of claim 17, wherein the small molecule inhibitor is batimastat.

19. (canceled)

20. The method of claim 12, further comprising administering to the subject anti-retroviral therapy (ART) or highly active anti-retroviral therapy (HAART).

21. A composition comprising a therapeutically effective amount of an inhibitor of CD62L shedding and an anti-retroviral agent.

22. A method of inducing human immunodeficiency virus (HIV) release from infected cells, comprising contacting the cells with an agent that induces CD62L shedding.
